(19) United States
(12) Patent Application Publication
(10) Pub. No.: US 2026/0209196 A1
(43) Pub. Date: Jul. 23, 2026

(54) AMIDINOUREA DERIVATIVES AND THEIR USE IN THE TREATMENT OF CANCER

(71) Applicants: UNIVERSITE COTE D'AZUR, NICE (FR); CENTRE NATIONAL DE LA RECHERCHE SCIENTIFIQUE, PARIS (FR); INSTITUT NATIONAL DE LA SANTÉ ET DE LA RECHERCHE MÉDICALE, PARIS (FR)

(72) Inventors: Rachid BENHIDA, NICE (FR); Cyril RONCO, NICE (FR); Nedra TEKAYA-HAMOUDA, SAINT-LAURENT-DU-VAR (FR); Thomas BOTTON, NICE (FR); Oleksandr GRYTSAI, MACCLESFIELD (GB); Stéphane ROCCHI

(73) Assignees: UNIVERSITE COTE D'AZUR, NICE (FR); CENTRE NATIONAL DE LA RECHERCHE SCIENTIFIQUE, PARIS (FR); INSTITUT NATIONAL DE LA SANTÉ ET DE LA RECHERCHE MÉDICALE, PARIS (FR)

(21) Appl. No.: 19/140,736

(22) PCT Filed: Dec. 22, 2023
(86) PCT No.: PCT/EP2023/087546

§ 371 Date: Jun. 18, 2025

(30) Foreign Application Priority Data
Dec. 22, 2022 (EP) 22307010.3

(51) Int. Cl. C07D 277/82; A61K 31/426; A61K 31/428; A61P 35/00; A61P 35/02; C07D 277/48

(52) U.S. Cl. CPC C07D 277/82; A61K 31/426; A61K 31/428; A61P 35/00; A61P 35/02; C07D 277/48

(57) ABSTRACT

Amidinourea derivatives and the use of these compounds in a method for treating cancer, in particular melanoma.

### TECHNICAL FIELD

The present disclosure relates to amidinourea derivatives. The present disclosure also relates to the use of these compounds in a method for treating cancer, in particular melanoma.

### BACKGROUND

Cancers represent one of the most important causes of death in France. Among cancers, melanoma is a skin cancer that is widespread in France, with about 16,000 new cases diagnosed each year and more than 2,100 deaths. This cancer is therefore a major public health problem. Melanoma is a malignant tumor developed from melanocytes that are responsible for the synthesis of melanin, which are photoprotective pigments. Melanoma is an extremely aggressive tumor with a high metastatic potential towards lymph nodes, liver, lungs, central nervous system and skin. As soon as metastases appear, the vital prognosis becomes unfavorable because of the poor efficiency of all current treatments.

Recently, encouraging results have been obtained with BRAF inhibitors (vemurafenib (PLX 4032), dabrafenib and encorafenib), which target only B-Raf mutant melanomas (approximately 50% of the metastatic melanoma) alone or in combination with MEK inhibitors (trametinib, selumetinib, cobimetinib). Unfortunately, after a short period of remission, melanoma, in almost all cases, gains resistance against these drugs and metastases develop again, only increasing the patient's life expectancy by about 2 months. Immunotherapies have also recently been developed. They are based on anti-CTLA4 and/or Anti-PD1 antibodies that reactivate the immune response. However, immunotherapies give an objective response in only 15 to 30% of patients.

Thus, there is a need for compounds and compositions with anti-proliferative properties, which could be used in particular to treat patients with melanoma and for example, patients with BRAF inhibitor-resistant melanoma.

### SUMMARY

The inventors surprisingly found that amidinourea derivatives comprising a heteroaryl moiety, have a high biological activity towards cancer lines such as melanoma cell lines, including melanoma cell lines resistant to BRAF inhibitors.

Consequently, in a first aspect, the disclosure relates to a compound of formula (I)

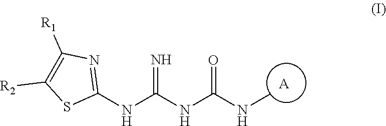

wherein
R₁ and R₂ are independently selected from H, C₁-C₆ alkyl, C₃-C₆ cycloalkyl, C₁-C₆ haloalkyl, C₂-C₆ alkenyl, C₂-C₆ alkynyl, heterocyclyl having 5 to 10 ring atoms, aryl having 6 to 10 ring atoms, heteroaryl having 5 to 10 ring atoms, and C₇-C₁₆ aralkyl, said alkyl, cycloalkyl, haloalkyl, alkenyl, alkynyl, heterocyclyl, aryl, heteroaryl and aralkyl being optionally substituted with one or more substituents preferably selected from oxo, halogen, C₁-C₆ alkyl, C₁-C₆ alkoxy, C₁-C₆ haloalkyl, —OH, —OR, —NR′R″, —NO₂, —CN and —(CO)—R;
or R₁ and R₂, together with the carbon-carbon double bond between them, form a 6 to 10 membered aryl or heteroaryl ring, said aryl and heteroaryl being optionally substituted with one or more substituents preferably selected from oxo, halogen, C₁-C₆ alkyl, C₁-C₆ alkoxy, C₁-C₆ haloalkyl, —OH, —OR, —NR′R″, —NO₂, —CN and —(CO)—R;
Ring A is selected from aryl having 6 to 10 ring atoms, heteroaryl having 5 to 10 ring atoms, C₃-C₆ cycloalkyl, and heterocyclyl having 5 to 10 ring atoms, said aryl, heteroaryl, cycloalkyl and heterocyclyl being optionally substituted with one or more substituents preferably selected from oxo, halogen, C₁-C₆ alkyl, C₁-C₆ alkenyl, C₁-C₆ alkynyl, C₁-C₆ alkoxy, C₁-C₆ haloalkyl, —OH, —OR, —NR′R″, —NO₂, —CN and —(CO)—R;
each R is independently selected from H, C₁-C₆ alkyl, OH, C₁-C₆ alkoxy and —NR′R″; and
each R′ and R″ is independently selected from H and C₁-C₆ alkyl, and pharmaceutically acceptable salts thereof.

In another aspect, the disclosure relates to a pharmaceutical composition comprising a compound of the disclosure, and a pharmaceutically acceptable carrier.

In another aspect, the disclosure relates to a compound of the disclosure for use in a method for treating cancer.

In another aspect, the disclosure relates to a method for treating cancer, said method comprising administering to a subject a therapeutically efficient amount of a compound of the disclosure or a pharmaceutical composition as described herein.

In another aspect, the disclosure relates to the use of a compound of the disclosure, for the manufacture of a medicament for the treatment of cancer.

In another aspect, the disclosure relates to the use of a compound of the disclosure for use as a drug.

### DETAILED DESCRIPTION

### Definitions

As used herein, the terms “C₁-C₆ alkyl”, by itself or as part of another substituent, refer to a linear or branched alkyl functional group having 1 to 6 carbon atoms. Suitable alkyl groups include methyl, ethyl, n-propyl, i-propyl, n-butyl, i-butyl, s-butyl and t-butyl, pentyl and its isomers (e.g. n-pentyl, iso-pentyl), and hexyl and its isomers (e.g. n-hexyl, iso-hexyl).

As used herein, the terms “C₃-C₆ cycloalkyl” refer to a saturated or unsaturated cyclic group having 3 to 6 carbon atoms. Suitable cycloalkyl groups include cyclopropyl, cyclobutyl, cyclopentyl and cyclohexyl.

As used herein, the term “halogen” refers to a fluoro (—F), chloro (—CI), bromo (—Br), or iodo (—I) group.

As used herein, the terms “C₁-C₆ haloalkyl” refer to a C₁-C₆ alkyl as defined herein that is substituted by one or more halogen group as defined herein. Suitable C₁-C₆ haloalkyl groups include trifluoromethyl and dichloromethyl.

As used herein, the terms “C₂-C₆ alkenyl” refer to a straight or branched hydrocarbon moiety having at least one carbon-carbon double bond. Alkenyl groups include, for example, ethenyl (i.e., vinyl), propenyl, butenyl, 1-methyl-2-buten-1-yl, pentenyl, hexenyl, octenyl, and butadienyl.

As used herein, the terms “C₂-C₆ alkynyl” refer to a straight or branched hydrocarbon moiety having at least one carbon-carbon triple bond. Examples of “alkynyl” include ethynyl, 2-propynyl (propargyl), 1-propynyl, pentynyl, hexynyl, and allenyl groups, and the like.

As used herein, the terms “C₁-C₆ alkoxy” refer to a —O-alkyl group, wherein the alkyl group is a C₁-C₆ alkyl as defined herein. Suitable C₁-C₆ alkoxy groups include methoxy, ethoxy, propoxy.

As used herein, the terms “aryl having 6 to 10 ring atoms” refer to a polyunsaturated, aromatic hydrocarbyl group having a single ring or multiple aromatic rings fused together, containing 6 to 10 ring atoms, wherein at least one ring is aromatic. The aromatic ring may optionally include one to two additional rings (cycloalkyl, heterocyclyl or heteroaryl as defined herein) fused thereto. Suitable aryl groups include phenyl, naphtyl and phenyl ring fused to a heterocyclyl, like benzopyranyl, benzodioxolyl, benzodioxanyl and the like.

As used herein, the terms “heteroaryl having 5 to 10 ring atoms” refer to a polyunsaturated, aromatic ring system having a single ring or multiple aromatic rings fused together or linked covalently, containing 5 to 10 atoms, wherein at least one ring is aromatic and at least one ring atom is a heteroatom selected from N, O and S. The nitrogen and sulfur heteroatoms may optionally be oxidized and the nitrogen heteroatoms may optionally be quaternized. Such rings may be fused to an aryl, cycloalkyl or heterocyclyl ring. Non-limiting examples of such heteroaryl, include: furanyl, thiophenyl, pyrrolyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, indolyl, isoindolyl, benzofuranyl, isobenzofuranyl, benzothiophenyl, isobenzothiophenyl, indazolyl, benzimidazolyl, benzoxazolyl, purinyl, benzothiadiazolyl, quinolinyl, isoquinolinyl, cinnolinyl, quinazolinyl and quinoxalinyl.

As used herein, the terms “heterocyclyl having 5 to 10 ring atoms” refer to a saturated or unsaturated cyclic group having 5 to 10 ring atoms, wherein at least one ring atom is a heteroatom selected from N, O and S. The nitrogen and sulfur heteroatoms may optionally be oxidized and the nitrogen heteroatoms may optionally be quaternized. Examples of heterocycle include, but are not limited to, tetrahydropyridyl, piperidinyl, morpholinyl, tetrahydrofuranyl, tetrahydrothienyl, piperazinyl, 1-azepanyl, imidazolinyl, 1,4-dioxanyl and the like.

As used herein, the terms “C₇-C₁₆ aralkyl” refer to an alkyl group as defined herein that is substituted by one or more aryl groups as defined herein. Aralkyl groups include, for example, benzyl groups.

Various embodiments of the disclosure are described herein. It will be recognized that features specified in each embodiment may be combined with other specified features to provide further embodiments.

The present disclosure encompasses the compounds of the disclosure their tautomers, enantiomers, diastereomers, racemates or mixtures thereof, and their hydrates, solvates or pharmaceutically acceptable salts.

The terms “pharmaceutically acceptable salts” refers to salts that retain the biological effectiveness and properties of the compounds of this disclosure and, which typically are not biologically or otherwise undesirable.

Any formula given herein is also intended to represent unlabeled as well as isotopically forms of the compounds, like deuterium labeled compounds or ¹⁴C-labeled compounds.

### Compound of Formula (I)

The present disclosure relates to a compound of formula (I)

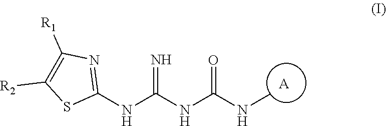

wherein
R₁ and R₂ are independently selected from H, C₁-C₆ alkyl, C₃-C₆ cycloalkyl, C₁-C₆ haloalkyl, C₂-C₆ alkenyl, C₂-C₆ alkynyl, heterocyclyl having 5 to 10 ring atoms, aryl having 6 to 10 ring atoms, heteroaryl having 5 to 10 ring atoms, and C₇-C₁₆ aralkyl, said alkyl, cycloalkyl, haloalkyl, alkenyl, alkynyl, heterocyclyl, aryl, heteroaryl and aralkyl being optionally substituted with one or more substituents preferably selected from oxo, halogen, C₁-C₆ alkyl, C₁-C₆ alkoxy, C₁-C₆ haloalkyl, —OH, —OR, —NR′R″, —NO₂, —CN and —(CO)—R;
or R₁ and R₂, together with the carbon-carbon double bond between them, form a 6 to 10 membered aryl or heteroaryl ring, said aryl and heteroaryl being optionally substituted with one or more substituents preferably selected from oxo, halogen, C₁-C₆ alkyl, C₁-C₆ alkoxy, C₁-C₆ haloalkyl, —OH, —OR, —NR′R″, —NO₂, —CN and —(CO)—R;
Ring A is selected from aryl having 6 to 10 ring atoms, heteroaryl having 5 to 10 ring atoms, C₃-C₆ cycloalkyl, and heterocyclyl having 5 to 10 ring atoms, said aryl, heteroaryl, cycloalkyl and heterocyclyl being optionally substituted with one or more substituents preferably selected from oxo, halogen, C₁-C₆ alkyl, C₁-C₆ alkenyl, C₁-C₆ alkynyl, C₁-C₆ alkoxy, C₁-C₆ haloalkyl, —OH, —OR, —NR′R″, —NO₂, —CN and —(CO)—R;
each R is independently selected from H, C₁-C₆ alkyl, OH, C₁-C₆ alkoxy and —NR′R″; and
each R′ and R″ is independently selected from H and C₁-C₆ alkyl, and pharmaceutically acceptable salts thereof.

In one embodiment, Ring A is selected from aryl having 6 to 10 ring atoms, heteroaryl having 5 to 10 ring atoms, C₄-C₆ cycloalkyl, and heterocyclyl having 5 to 10 ring atoms, said aryl, heteroaryl, cycloalkyl and heterocyclyl being optionally substituted with one or more substituents preferably selected from oxo, halogen, C₁-C₆ alkyl, C₁-C₆ alkenyl, C₁-C₆ alkynyl, C₁-C₆ alkoxy, C₁-C₆ haloalkyl, —OH, —OR, —NR′R″, —NO₂, —CN and —(CO)—R.

In one embodiment, Ring A is selected from aryl having 6 to 10 ring atoms and C₄-C₆ cycloalkyl, said aryl and cycloalkyl being optionally substituted with one or more substituents preferably selected from oxo, halogen, C₁-C₆ alkyl, C₁-C₆ alkenyl, C₁-C₆ alkynyl, C₁-C₆ alkoxy, C₁-C₆ haloalkyl, —OH, —OR, —NR′R″, —NO₂, —CN and —(CO)—R.

In one embodiment, Ring A is an aryl having 6 to 10 ring atoms or a heteroaryl having 5 to 10 ring atoms, said aryl and heteroaryl being optionally substituted with one or more substituents preferably selected from oxo, halogen, C₁-C₆ alkyl, C₁-C₆ alkoxy, C₁-C₆ haloalkyl, —OH, —NR′R″, —NO₂, —CN and —(CO)—R.

In one embodiment, Ring A is a phenyl or a naphthyl, said phenyl and naphthyl being optionally substituted with one or more substituents preferably selected from halogen, C₁-C₆ alkyl, C₁-C₆ alkoxy, and —NO₂.

In one embodiment, R₁ and R₂ are independently selected from H, C₁-C₆ alkyl, and aryl having 6 to 10 ring atoms, said alkyl, and aryl, being optionally substituted with one or more substituents preferably selected from halogen, C₁-C₆ alkyl, and —NO₂;

or, R₁ and R₂, together with the carbon-carbon double bond between them, form a 6 to 10 membered aryl, said aryl being optionally substituted with one or more substituents preferably selected from halogen, C₁-C₆ alkyl, and —NO₂.

In one embodiment, the compound of formula (I) is not

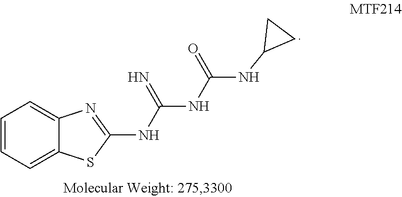 [SMILES: N=C(NC(=O)NC1CC1)Nc1nc2ccccc2s1]

In one embodiment, the compound of formula (I) is a compound of formula (II), (III) or (IV)

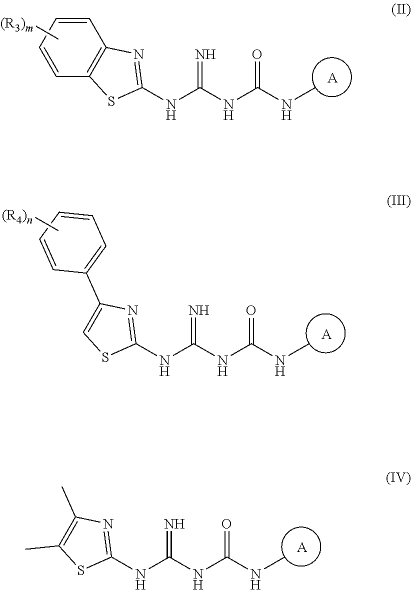

wherein
m is an integer between 0 and 5, preferably m is 0 or 1;
each R₃ is independently selected from halogen, C₁-C₆ alkyl, C₁-C₆ alkoxy, C₁-C₆ haloalkyl, —OH, —NR′R″, —NO₂, —CN and —(CO)—R, preferably selected from halogen, C₁-C₆ alkyl, C₁-C₆ alkoxy, and —NO₂;
n is an integer between 0 and 5, preferably n is 0 or 1; and
each R₄ is independently selected from halogen, C₁-C₆ alkyl, C₁-C₆ alkoxy, C₁-C₆ haloalkyl, —OH, —NR′R″, —NO₂, —CN and —(CO)—R, preferably selected from halogen, C₁-C₆ alkyl, C₁-C₆ alkoxy, and —NO₂.

In one embodiment. m is 0 or 1, and when m is 1, R₃ is —NO₂.

In one embodiment, n is 0 or 1, and when n is 1, R₄ is —NO₂.

In one embodiment, the compound of formula (I) is a compound of formula (V) or (VI)

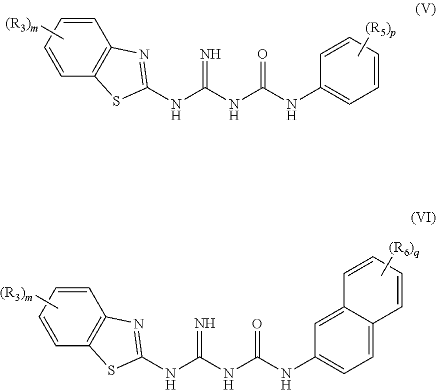

wherein
m is an integer between 0 and 5, preferably m is 0 or 1;
each R₃ is independently selected from halogen, C₁-C₆ alkyl, C₁-C₆ alkoxy, C₁-C₆ haloalkyl, —OH, —NR′R″, —NO₂, —CN and —(CO)—R, preferably selected from halogen, C₁-C₆ alkyl, C₁-C₆ alkoxy, and —NO₂;
p is an integer between 0 and 5, preferably p is 0, 1, 2, or 3;
each R₅ is independently selected from halogen, C₁-C₆ alkyl, C₁-C₆ alkoxy, C₁-C₆ haloalkyl, —OH, —NR′R″, —NO₂, —CN and —(CO)—R, preferably selected from halogen, C₁-C₆ alkyl, C₁-C₆ alkoxy, and —NO₂;
q is an integer between 0 and 7, preferably q is 0; and
each R₆ is independently selected from halogen, C₁-C₆ alkyl, C₁-C₆ alkoxy, C₁-C₆ haloalkyl, —OH, —NR′R″, —NO₂, —CN and —(CO)—R, preferably selected from halogen, C₁-C₆ alkyl, C₁-C₆ alkoxy, and —NO₂.

In one embodiment, the compound of formula (I) is a compound of formula (V) or (VI) and Ring A is a phenyl or a naphthyl, said phenyl and naphthyl being optionally substituted with one or more substituents preferably selected from halogen, C₁-C₆ alkyl, C₁-C₆ alkoxy, and —NO₂.

In another embodiment, the compound of formula (I) is a compound of formula (VII) or (VIII)

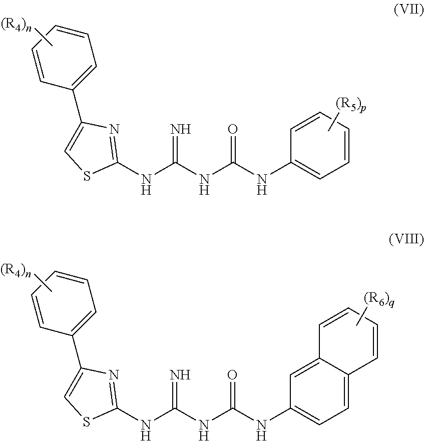

wherein
n is an integer between 0 and 5, preferably n is 0 or 1; and
each R₄ is independently selected from halogen, C₁-C₆ alkyl, C₁-C₆ alkoxy, C₁-C₆ haloalkyl, —OH, —NR′R″, —NO₂, —CN and —(CO)—R, preferably selected from halogen, C₁-C₆ alkyl, C₁-C₆ alkoxy, and —NO₂;
p is an integer between 0 and 5, preferably p is 0, 1, or 2;
each R₅ is independently selected from halogen, C₁-C₆ alkyl, C₁-C₆ alkoxy, C₁-C₆ haloalkyl, —OH, —NR′R″, —NO₂, —CN and —(CO)—R, preferably selected from halogen, C₁-C₆ alkyl, C₁-C₆ alkoxy, and —NO₂;
q is an integer between 0 and 7, preferably q is 0; and
each R₆ is independently selected from halogen, C₁-C₆ alkyl, C₁-C₆ alkoxy, C₁-C₆ haloalkyl, —OH, —NR′R″, —NO₂, —CN and —(CO)—R, preferably selected from halogen, C₁-C₆ alkyl, C₁-C₆ alkoxy, and —NO₂.

In another embodiment, the compound of formula (I) is a compound of formula (IX) or (X)

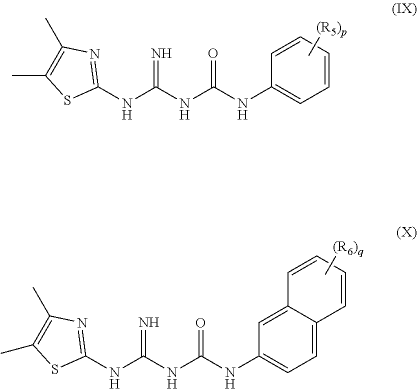

wherein
p is an integer between 0 and 5, preferably p is 0, 1, or 2;
each R₅ is independently selected from halogen, C₁-C₆ alkyl, C₁-C₆ alkoxy, C₁-C₆ haloalkyl, —OH, —NR′R″, —NO₂, —CN and —(CO)—R, preferably selected from halogen, C₁-C₆ alkyl, C₁-C₆ alkoxy, and —NO₂;
q is an integer between 0 and 7, preferably q is 0; and
each R₆ is independently selected from halogen, C₁-C₆ alkyl, C₁-C₆ alkoxy, C₁-C₆ haloalkyl, —OH, —NR′R″, —NO₂, —CN and —(CO)—R, preferably selected from halogen, C₁-C₆ alkyl, C₁-C₆ alkoxy, and —NO₂.

The present disclosure encompasses the compounds of the disclosure and their tautomers and it is well known that the amidinoureas can interconvert into different tautomers. Thus, the amidinoureas of formula (I), (II), (III), (IV), (V), (VI), (VII), (VIII), (IX) and (X) include the amidinourea as defined by the formula and all the corresponding tautomers. For example, for the compound of formula (I), the following tautomers can be considered as encompassed by formula (I):

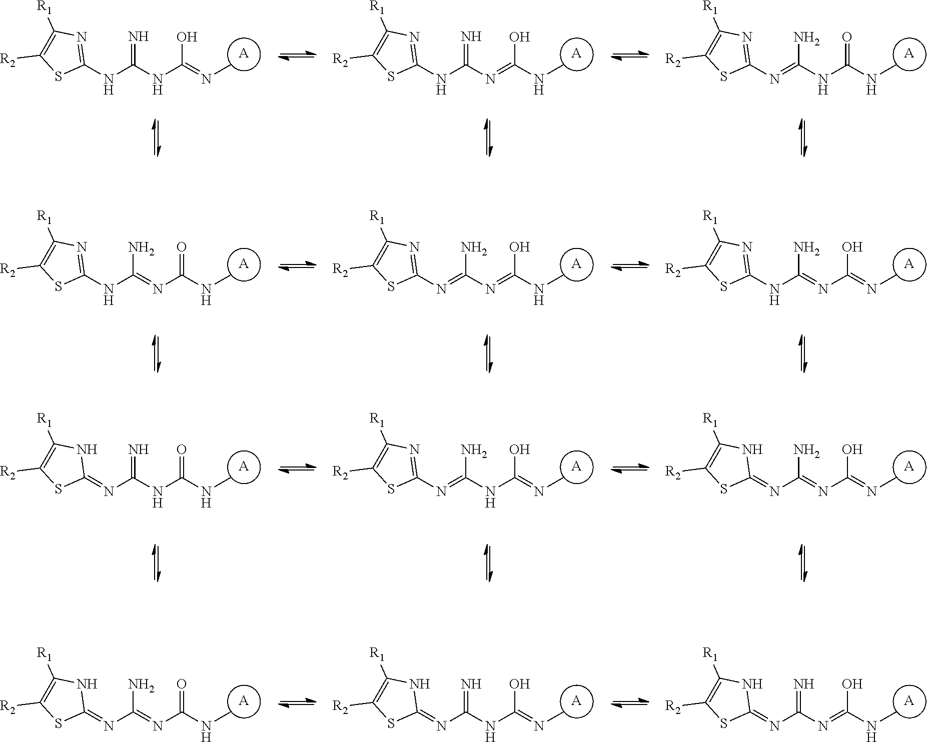

In one embodiment, the compound of formula (I) is selected from

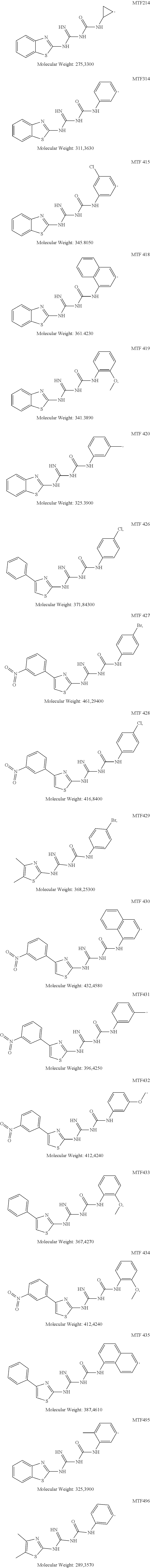

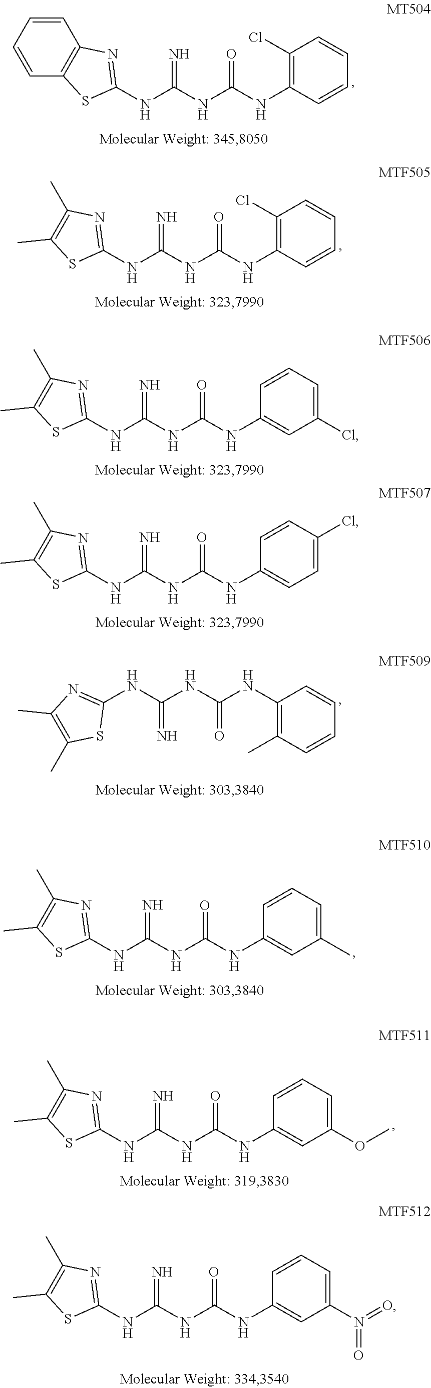

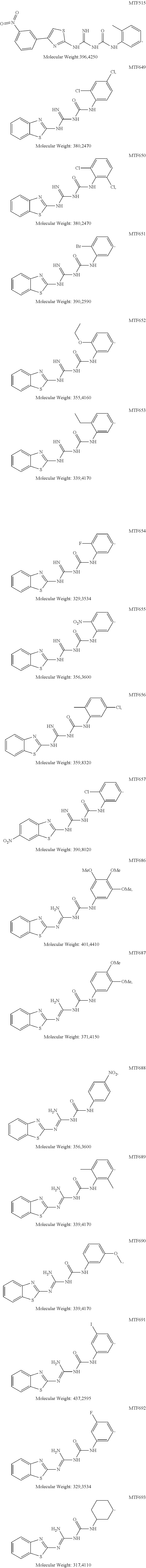

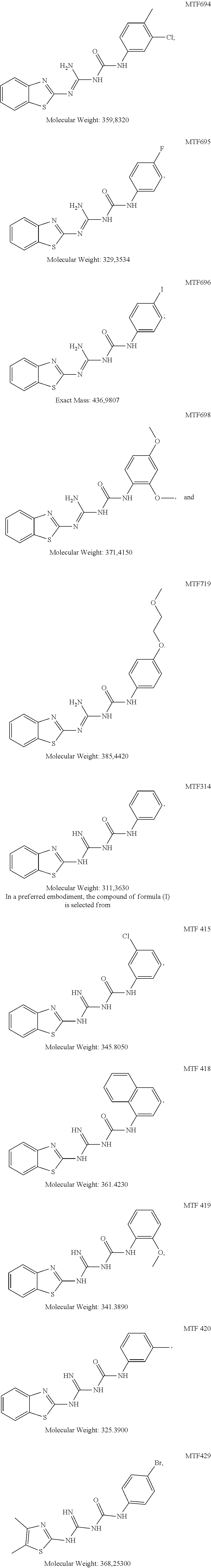

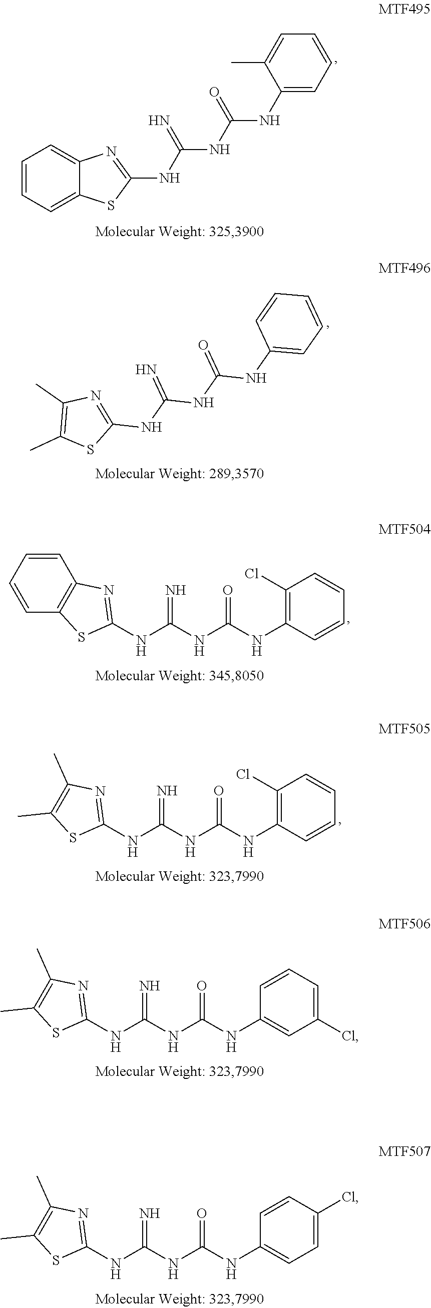 [SMILES: Cc1ccccc1NC(=O)NC(=N)Nc1nc2ccccc2s1 ; Cc1nc(NC(=N)NC(=O)Nc2ccccc2)sc1C ; N=C(NC(=O)Nc1ccccc1Cl)Nc1nc2ccccc2s1 ; Cc1nc(NC(=N)NC(=O)Nc2ccccc2Cl)sc1C ; Cc1nc(NC(=N)NC(=O)Nc2cccc(Cl)c2)sc1C ; Cc1nc(NC(=N)NC(=O)Nc2ccc(Cl)cc2)sc1C]

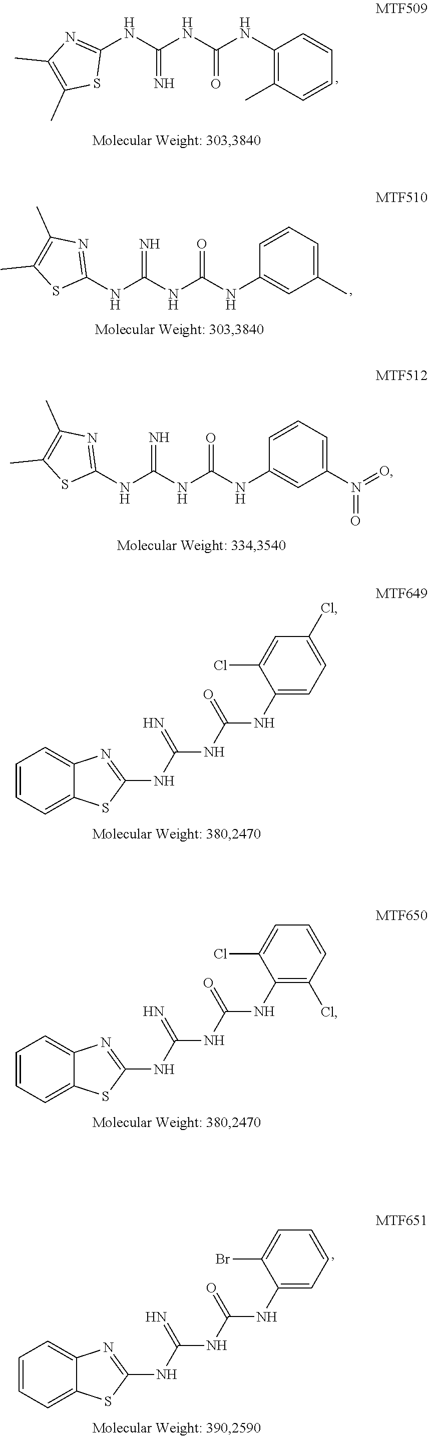

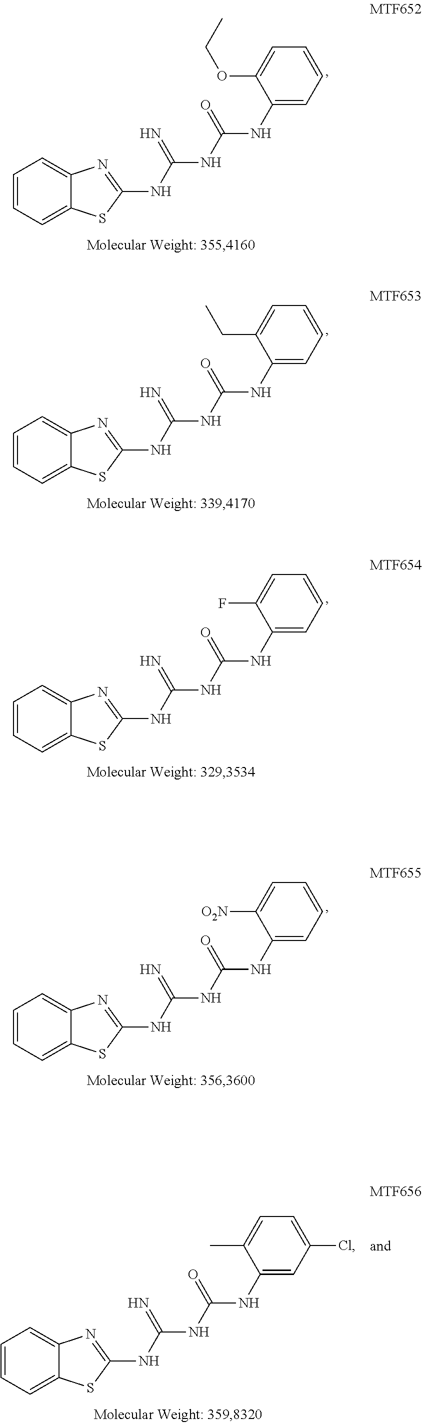 [SMILES: CCOc1ccccc1NC(=O)NC(=N)Nc1nc2ccccc2s1 ; CCc1ccccc1NC(=O)NC(=N)Nc1nc2ccccc2s1 ; N=C(NC(=O)Nc1ccccc1F)Nc1nc2ccccc2s1 ; N=C(NC(=O)Nc1ccccc1[N+](=O)[O-])Nc1nc2ccccc2s1 ; Cc1ccc(Cl)cc1NC(=O)NC(=N)Nc1nc2ccccc2s1]

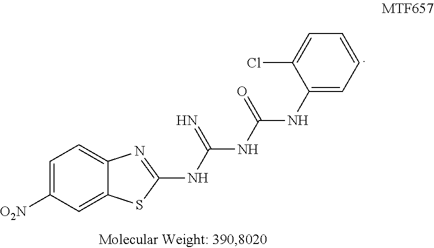 [SMILES: N=C(NC(=O)Nc1ccccc1Cl)Nc1nc2ccc([N+](=O)[O-])cc2s1]

In some embodiments, the compounds of the disclosure as described herein have anti-proliferative activity towards melanoma cell lines. Accordingly, they may advantageously be used in a method for treating cancer, and melanoma.

### Pharmaceutical Composition

The disclosure also relates to a pharmaceutical composition comprising a compound of the disclosure, and a pharmaceutically acceptable carrier.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. Suitable carrier include but are not limited to stabilizing agents, wetting and emulsifying agents, salts for varying osmolality, encapsulating agents, pH buffering substances, and buffers.

The form of the pharmaceutical compositions, the route of administration, the dosage and the regimen naturally depend upon the condition to be treated, the severity of the illness, the age, weight, and sex of the patient, etc.

The pharmaceutical compositions of the disclosure can be formulated for a topical, oral, intranasal, intraocular, intravenous, intramuscular or subcutaneous administration and the like.

The pharmaceutical compositions can take the form of tablets, pills, capsules, semisolids, powders, sustained release formulations, solutions, suspensions, emulsions, syrups, elixirs, aerosols, or any other appropriate compositions; and comprise at least one compound according to this disclosure.

Because of their ease in administration, tablets and capsules represent the most advantageous oral dosage unit form, in which case solid pharmaceutical carriers are obviously employed. If desired, tablets may be sugar coated or enteric coated by standard techniques. The tablets or pills can be coated to provide a dosage form affording the advantage of prolonged action. For example, the tablet or pills can comprise an inner dosage and an outer dosage component, the latter being in the form of an envelope over the former. The two components can be separated by an enteric layer, which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The compound of the disclosure and the further agent may also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, mixtures thereof and in oils. Under ordinary conditions of storage and use, these preparations contain a preservative to prevent the growth of microorganisms.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

Preferably, the pharmaceutical composition is suitable for parenteral administration. For parenteral administration in an aqueous solution, for example, the solution may be suitably buffered and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

For aerosol administration, the compound of the disclosure and the further agent are preferably supplied in finely divided from along with a surfactant and propellant. The surfactant must, of course, be nontoxic, and preferably soluble in the propellant. Representative of such agents are the esters or partial esters of fatty acids containing from 6 to 22 carbon atoms, such as caproic, octanoic lauric, palmitic, stearic, linoleic, linolenic, olesteric and oleic acids with an aliphatic polyhydric alcohol or its cyclic anhydride. Mixed esters, such as mixed or natural glycerides may be employed. A carrier can also be included, as desired, as with, e.g., lecithin for intranasal delivery. An example includes a solution in which each milliliter included 7.5 mg NaCl, 1.7 mg citric acid monohydrate, 3 mg disodium phosphate dihydrate and 0.2 mg benzalkonium chloride solution (50%) (Gozes et al., J Mol Neurosci. 19 (1-2): 167-70 (2002)).

Suitable compositions for topical application include aqueous solutions, suspensions, ointments, creams, gels or sprayable formulations, e.g. aerosol administration.

The doses used for the administration can be adapted as a function of various parameters, and in particular as a function of the mode of administration used, of the relevant pathology, or alternatively of the desired duration of treatment. It will be appreciated that appropriate dosages of the compounds, and compositions comprising the compounds, can vary from patient to patient. Determining the optimal dosage will generally involve the balancing of the level of therapeutic benefit against any risk or deleterious side effects of the treatments described herein. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

### Method of Use

The compounds of the disclosure exhibit valuable pharmaceutical properties as indicated in the in vitro provided in the examples and are therefore indicated for therapy.

The disclosure also relates to a compound of the disclosure for use as a medicament.

The disclosure also relates to a compound of the disclosure for use in a method for treating cancer.

As used herein, the term “cancer” has its general meaning in the art and includes an abnormal state or condition characterized by rapidly proliferating cell growth. The term is meant to include all types of cancerous growths or oncogenic processes, metastatic tissues or malignantly transformed cells, tissues or organs, irrespective of histopathologic type or stage of invasiveness. The term cancer includes malignancies of the various organ systems, such as affecting skin, lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

Examples of cancer include, but are not limited, to hematological malignancies such as B-cell lymphoid neoplasm, T-cell lymphoid neoplasm, non-hodgkin lymphoma (NHL), B-NHL, T-NHL, chronic lymphocytic leukemia (CLL), small lymphocytic lymphoma (SLL), mantle cell lymphoma (MCL), NK-cell lymphoid neoplasm, and myeloid cell lineage neoplasm. Examples of non-hematological cancers include, but are not limited to, skin cancer, colon cancer, breast cancer, lung cancer, brain cancer, prostate cancer, head and neck cancer, pancreatic cancer, bladder cancer, colorectal cancer, bone cancer, cervical cancer, liver cancer, oral cancer, esophageal cancer, thyroid cancer, kidney cancer, stomach cancer and testicular cancer.

In specific embodiment, the disclosure relates to a compound of the disclosure for use in a method for treating a cancer selected from leukemia, Non-Small Cell Lung Cancer (NSCLC), colon cancer, central nervous system (CNS) cancer, melanoma, ovarian cancer, renal cancer, prostate and breast cancer.

In one particular embodiment, the disclosure also relates to a compound of the disclosure for use in a method for treating cancer, and preferably BRAF inhibitor-resistant melanoma.

The disclosure relates to a method for treating cancer, said method comprising administering to a subject a therapeutically efficient amount of a compound of the disclosure or a pharmaceutical composition as described herein.

The terms “therapeutically efficient amount” of a compound refer to an amount of the compound that will elicit the biological or medical response of a subject, for example, ameliorate the symptoms, alleviate conditions, slow or delay disease progression, or prevent a disease. The therapeutically effective amount of the compound of the disclosure, or pharmaceutical composition that comprises it may vary according to factors such as the disease state, age, sex, and weight of the individual, and the ability of the product or pharmaceutical composition to elicit a desired response in the individual. Dosage regimens may be adjusted to provide the optimum therapeutic response. A therapeutically effective amount is also typically one in which any toxic or detrimental effect of the compound or pharmaceutical composition is outweighed by the therapeutically beneficial effects.

As used herein, the term “subject” denotes an animal, preferably a mammal. Preferably, a subject according to the disclosure is a human.

In the context of the disclosure, the term “treating” or “treatment”, as used herein, means reversing, alleviating or inhibiting the progress of a disease or condition, or reversing, alleviating or inhibiting the progress of one or more symptoms of a disorder or condition.

The compound of the present disclosure is generally administered according to known procedures, at dosages and for periods of time effective to induce a therapeutic effect in the patient. The administration can be systemic or local. Systemic administration is preferably parenteral such as subcutaneous (SC), intramuscular (IM), intravascular such as intravenous (IV) or intraarterial; intraperitoneal (IP); intradermal (ID), interstitial or else. The administration may be for example by injection or perfusion. The disclosure also relates to the use of a compound of the disclosure, for the manufacture of a medicament for the treatment of cancer. In one embodiment, cancer is melanoma. In one embodiment, cancer is BRAF inhibitor-resistant melanoma.

### FIGURES LEGENDS

FIG. 1 represents the data obtained from the NCI-60 Human Tumor Cell Lines Screen after 48 hours treatment with 10 UM of MTF504.

FIG. 2 shows the inhibition of A375 melanoma cell confluence measured by Incucyte in response to doses of MTF504 ranging from 100 nM to 3 M.

FIG. 3 shows the absence of toxicity of MTF504 on normal human melanocytes measured by Trypan blue counting.

FIG. 4 shows the comparison of the anti-melanoma effect of various amidinoureas showing increased biological activity at low concentrations (10 nM). Vemurafenib 5 μM was used as a positive control for cell death.

FIG. 5 shows the comparison of the anti-melanoma effect of the amidinourea MTF504 versus the previously described CRO15 and MTF319 after 24 hours of treatment. These results show that Amidinourea according to the invention are more efficient than previously reported compounds to decrease melanoma cell viability.

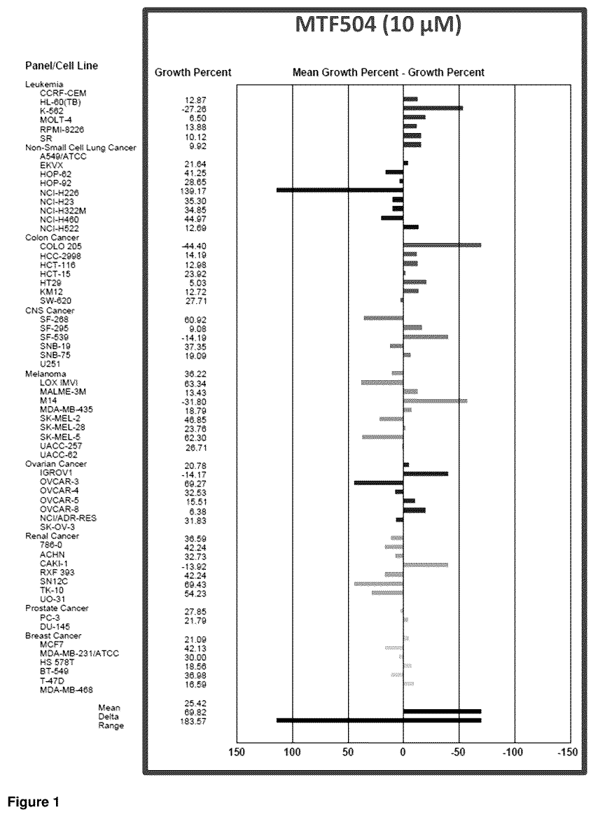

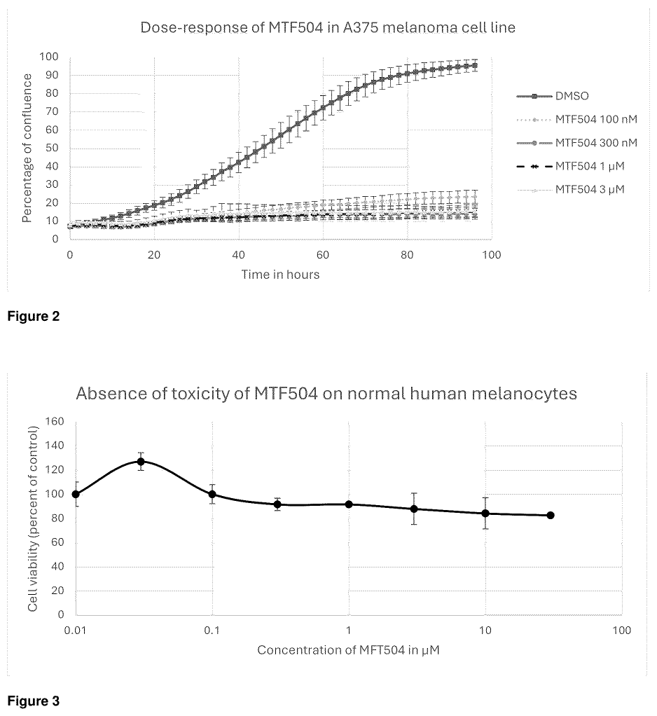

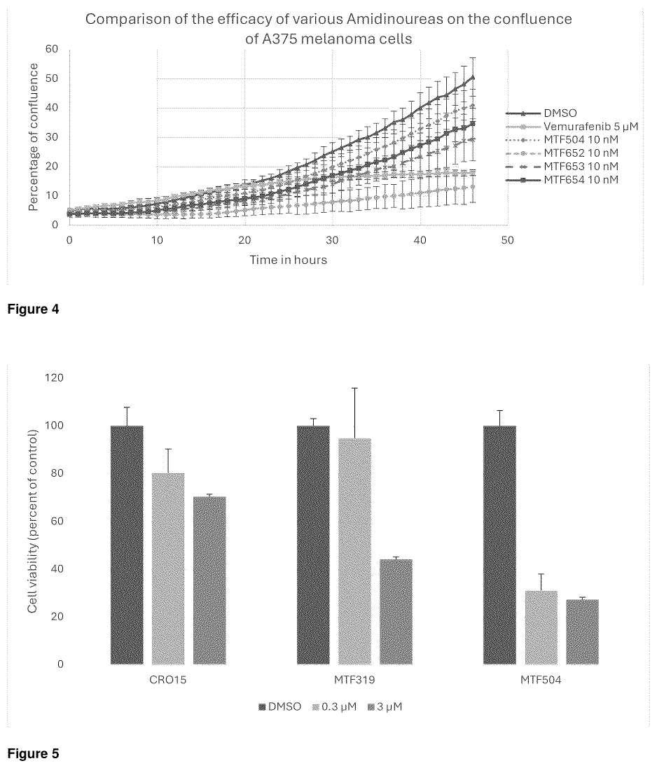

### EXAMPLES

### Chemical Synthesis and Characterization

Methanol, DMF, ethyl acetate, diethyl ether, cyclohexane and dichloromethane were purchased from Sigma Aldrich. DMF was dried by distillation under reduced pressure over MgSO₄, methanol, ethanol, diethyl ether, ethyl acetate and dichloromethane were used as received. All chemicals were purchased from Aldrich, Merck or Alfa Aesar and used without further purification. Thin layer chromatography (TLC) was performed on precoated Merck 60 GF254 silica gel plates and revealed first by visualization under UV light (254 nm and 360 nm) 1H and ¹³C NMR spectra were recorded on a Bruker Advance 200 MHz spectrometer or a Bruker Advance 400 MHz or a Bruker Advance 500 MHz. Mass spectra (ESI-MS) were recorded on a Bruker (Daltonics Esquire 3000+). HRMS spectra were recorded on a ThermoFisher Q Exactive (ESI-MS) at a resolution of 140 000 at m/z 200. The purity of compounds was further assayed by HPLC analysis on a JASCO PU-2089 apparatus with Supelco analytical column Ascentis Express C18, 100 mm×4.6 mm 5 μm. Flow: 1 mL/min. Eluent A: water with 1% % formic acid. Eluent B: CH₃CN with 1‰ formic acid. Two different methods were used which are described as follows. Method 1: 30% B for 1 min, 30% B to 100% B over 5 min, 100% B for 2.5 min then from 100% B to 30% B over 30 sec, 30% B for 7 min (16 min in total). Method 2: 30% B for 1 min, 30% B to 100% B over 5 min, 100% B for 20 min then from 100% B to 30% B over 1 min, 30% B for 3 min (30 min in total). Method 3: 30% B to 100% B over 6 min, 100% B for 8 min then from 100% B to 30% B over 1 min (16 min in total). Method 4: 50% B for 3 min, 50% B to 95% B over 2 min, 95% B for 10 min then from 95% B to 50% B over 2 min, 50% B for 3 min (20 min in total). Method 5: 5% B for 3 min, 50% B to 95% B over 12 min, 95% B for 5 min then from 95% B to 50% B over 2 min, 50% B for 3 min (total time: 25 min).

### Synthetic Procedures and Characterizations:

General Procedure (A). To a suspension of 4H-1,3,5-triazino[2,1-b]benzothiazol-4-one (1.0 eq.) in acetonitrile (25 mL) was added DBU (2.0 eq.) and corresponding amine (3.0 eq.). The reaction mixture was refluxed until the complete conversion of the starting material. The reaction mixture was cooled to r.t. and was concentrated under reduced pressure. Purification by silica gel column chromatography (Cyclohexane:EtOAc, 10:0 to 6:4) afforded the corresponding amide.

General procedure (B). To a solution of corresponding guanidine (1.0 eq.) in DMF (8 mL/mmol) was added corresponding isocyanate (1.0 eq.) and the mixture was stirred overnight at r.t. After completion of the reaction, the reaction mixture was poured into water (60 mL/mmol) and the precipitate was collected and washed with methanol (3×8 mL/mmol) and diethyl ether (2×8 mL/mmol).

N—(N-(benzo[d]thiazol-2-yl) carbamimidoyl)cyclopropylamine-1-carboxamide (MTF214) To a suspension of 4H-1,3,5-triazino[2,1-b]benzothiazol-4-one (300 mg, 1.37 mmol) in acetonitrile (25 mL) was added DBU (0.4 mL, 2.75 mmol), cyclopropylamine (0.29 mL, 4.13 mmol) and the mixture was refluxed overnight. The reaction mixture was cooled to r.t. and was concentrated under reduced pressure. Purification by silica gel column chromatography (Cyclohexane:EtOAc, 10:0 to 6:4) afforded the desired compound as a white powder (150 mg, 42%) M.p.=230° C. ¹H NMR (200 MHz, DMSO-d6): δ 9.57 (s, 1H), 9.26 (br. s, 1H), 8.81 (br. s, 1H), 7.77 (dd, J=7.8, 0.8 Hz, 1H), 7.58 (d, J=7.9 Hz, 1H), 7.33 (td, J=7.7, 1.3 Hz, 1H), 7.26-7.00 (m, 2H), 2.58 (s, 1H), 0.58 (d, J=43.1 Hz, 4H). ¹³C NMR (126 MHz, DMSO-d6): δ 172.69, 155.82, 153.68, 151.36, 130.62, 125.71, 122.71, 121.18, 119.38, 22.17, 6.17 (2C). HRMS-ESI (m/z): [M+H]+ calc. for C₁₂H₁₄N₅OS⁺, 276.09136; Found: 276.09140. HPLC (λ₂₅₄): Purity 99.6%; t_{R}: 3.792 min (method 1).

N—(N-(benzo[d]thiazol-2-yl) carbamimidoyl)-anilinocarboxamide (MTF314) Synthesized following the general procedure A using 4H-1,3,5-triazino[2,1-b]benzothiazol-4-one (300 mg, 1.37 mmol), DBU (0.4 mL, 2.75 mmol) and aniline (0.55 mL, 4.15 mmol) to afford the desired compound as a white powder (136 mg, 32%). M.P.=287° C. ¹H NMR (400 MHz, DMSO-d6): δ 9.69 (br. s, 1H), 9.48 (br. s, 2H), 8.68 (br. s, 1H), 7.80 (d, J=7.5 Hz, 1H), 7.63 (d, J=7.7 Hz, 1H), 7.46 (s, 2H), 7.35 (d, J=6.8 Hz, 3H), 7.20 (t, J=7.2 Hz, 1H), 7.13-6.99 (m, 1H). ¹³C NMR (101 MHz, DMSO-d6): δ 172.21, 153.45, 152.52, 151.18, 138.07, 130.82, 128.92 (2C), 125.69, 123.22, 122.81, 121.15, 119.55, 119.06 (2C). HRMS-ESI (m/z): [M−H]⁻ calc, for C₁₅H₁₄N₅OS, 312.09143; Found: 312.09136. HPLC (λ₂₈₀): Purity 99.4%; t_{R}: 10.483 min (method 1).

N-benzo[d]thiazol-2-yl-N″-(3-chloroaniline-carbonyl)-guanidine (MTF415) Synthesized following the general procedure B using 1-(benzo[d]thiazol-2-yl) guanidine (1 g, 5.20 mmol) and 3-chlorophenyl isocyanate (0.63 mL, 5.20 mmol) to afford the title compound as an white powder (1.13 g, 63%). ¹H NMR (200 MHz, DMSO-d6): δ 9.82 (br. s, 1H), 9.62 (br. s, 1H), 9.27 (br. s, 1H), 8.65 (br. s, 1H), 7.82 (d, J=7.6 Hz, 1H), 7.73 (s, 1H), 7.64 (d, J=7.8 Hz, 1H), 7.36 (t, J=6.9 Hz, 3H), 7.27-7.08 (m, 2H). ¹³C NMR (50 MHz, DMSO-d6): δ 170.64, 153.57 (2C), 150.98, 139.89, 133.36, 131.02, 130.46, 125.74, 122.90, 122.74, 121.18, 119.66, 118.49, 117.46. HPLC (λ₂₈₀): Purity 100.0%; t_{R}: 21.800 min (method 2).

N-benzo[d]thiazol-2-yl-N″-(naphtalen-1-amine-carbonyl)-guanidine (MTF418) Synthesized following the general procedure B using 1-(benzo[d]thiazol-2-yl) guanidine (1 g, 5.20 mmol) and 1-naphthyl isocyanate (0.75 mL, 5.20 mmol) to afford the title compound as an white powder (1.07 g, 57%). ¹H NMR (400 MHz, DMSO-d6): δ 10.38 (s, 1H), 9.88 (s, 1H), 9.42 (br. s, 1H), 8.44 (br. s, 1H), 8.14 (d, J=8.5 Hz, 1H), 8.05 (d, J=7.3 Hz, 1H), 7.99 (d, J=8.0 Hz, 1H), 7.85 (d, J=7.6 Hz, 1H), 7.74 (d, J=8.0 Hz, 1H), 7.71-7.56 (m, 3H), 7.53 (t, J=7.9 Hz, 1H), 7.37 (t, J=7.4 Hz, 1H), 7.23 (t, J=7.5 Hz, 1H). ¹³C NMR (50 MHz, DMSO-d6): δ 171.93, 153.49, 152.50, 151.25, 133.72, 132.98, 130.93, 128.59, 126.26, 126.14, 125.85 (3C), 124.13, 123.04, 121.30, 121.13, 119.78, 117.94. HPLC (λ₂₈₀): Purity 96.8%; t_{R}: 12.067 min (method 2).

N-benzo[d]thiazol-2-yl-N″-(2-methoxyaniline-carbonyl)-guanidine (MTF419) Synthesized following the general procedure B using 1-(benzo[d]thiazol-2-yl) guanidine (1 g, 5.20 mmol) and 2-methoxyphenyl isocyanate (0.69 mL, 5.20 mmol) to afford the title compound as an white powder (815 mg, 46%). ¹H NMR (400 MHz, DMSO-d6): δ 10.46 (br. s, 1H), 9.72 (s, 1H), 9.40 (br. s, 1H), 8.14 (br. s and d, J=7.6 Hz, 2H), 7.85 (d, J=7.8 Hz, 1H), 7.64 (d, J=7.8 Hz, 1H), 7.37 (t, J=7.5 Hz, 1H), 7.23 (t, J=7.4 Hz, 1H), 7.05 (dd, J=19.1, 7.6 Hz, 2H), 6.93 (t, J=7.2 Hz, 1H), 3.96 (s, 3H). ¹³C NMR (50 MHz, DMSO-d6) δ 171.76, 153.08, 151.58, 151.20, 148.22, 130.96, 127.40, 125.81, 123.20, 123.04, 121.33, 120.56, 119.74, 118.86, 110.91, 55.86. HPLC (λ₂₈₀): Purity 97.5%; t_{R}: 11.317 min (method 2).

N-benzo[d]thiazol-2-yl-N″-(3-methylaniline-carbonyl)-guanidine (MTF420) Synthesized following the general procedure B using 1-(benzo[d]thiazol-2-yl) guanidine (1 g, 5.20 mmol) and m-tolyl isocyanate (0.67 mL, 5.20 mmol) to afford the title compound as an white powder (1.32 g, 78%). ¹H NMR (400 MHz, DMSO-d6): δ 9.68 (s, 1H), 9.41 (s, 2H), 8.69 (s, 1H), 7.80 (d, J=7.6 Hz, 1H), 7.63 (d, J=8.0 Hz, 1H), 7.38-7.33 (m, 1H), 7.30 (s, 1H), 7.28-7.17 (m, 3H), 6.89 (d, J=6.8 Hz, 1H), 2.30 (s, 3H). ¹³C NMR (50 MHz, DMSO-d6); δ 172.35, 153.48, 152.56, 151.20, 138.26, 138.00, 130.81, 128.81, 125.74, 123.99, 122.86, 121.21, 119.56 (2C), 116.20, 21.13. HPLC (λ₂₈₀): Purity 98.7%; t_{R}: 11.450 min (method 2).

N-4-phenylthiazol-2-yl-N″-(4-chloroaniline-carbonyl)-guanidine (MTF426) Synthesized following the general procedure B using 1-(4-phenylthiazol-2-yl) guanidine (1 g, 4.58 mmol) and 4-chlorophenyl isocyanate (702 mg, 4.58 mmol) to afford the title compound as a white powder (799 mg, 47%). ¹H NMR (400 MHz, DMSO-d6): δ10.77 (s, 1H), 9.75 (s, 1H), 9.38 (s, 1H), 8.37 (s, 1H), 7.91 (d, J=7.2 Hz, 2H), 7.50 (s, 3H), 7.45-7.34 (m, 4H), 7.31 (t, J=7.3 Hz, 1H). ¹³C NMR (50 MHz, DMSO-d6): δ 152.53 (2C), 149.64, 137.57, 134.32, 128.66 (5C), 127.63, 126.48, 125.65 (2C), 120.48 (2C), 106.55. HPLC (λ₂₅₄): Purity 95.1%; t_{R}: 11.808 min (method 2).

N-4-(3-nitrophenyl) thiazol-2-yl-N″-(4-bromoaniline-carbonyl)-guanidine (MTF427) Synthesized following the general procedure B using 1-(4-(3-nitrophenyl) thiazol-2-yl) guanidine (1 g, 3.80 mmol) and 4-bromophenyl isocyanate (0.746 mg, 3.77 mmol) to afford the title compound as a white powder (1.09 g, 63%). ¹H NMR (400 MHz, DMSO-d6): δ 10.90 (s, 1H), 9.64 (s, 1H), 9.42 (s, 1H), 9.01 (s, 1H), 8.63 (s, 1H), 8.33 (d, J=7.9 Hz, 1H), 8.11 (dd, J=8.1, 1.5 Hz, 1H), 7.79 (s, 21H), 7.67 (t, J=8.0 Hz, 1H), 7.47 (s, 4H). ¹³C NMR (50 MHz, DMSO-d6): δ 152.52 (2C), 148.32, 147.20, 138.06 (2C), 135.86, 131.87, 131.55 (2C), 130.14, 122.03, 120.84 (2C), 119.85, 114.34, 109.42. HPLC (λ₂₈₀): Purity 100.0%; t_{R}: 12.033 min (method 2).

N-4-(3-nitrophenyl) thiazol-2-yl-N″-(4-chloroaniline-carbonyl)-guanidine (MTF428) Synthesized following the general procedure B using 1-(4-(3-nitrophenyl) thiazol-2-yl) guanidine (1 g, 3.80 mmol) and 4-chlorophenyl isocyanate (584 mg, 3.80 mmol) to afford the title compound as an yellowish powder (910 mg, 58%). ¹H NMR (400 MHz, DMSO-d6) δ 10.88 (s, 1H), 9.58 (s, 1H), 9.39 (s, 1H), 8.59 (s, 1H), 8.42 (br.s, 1H), 8.29 (d, J=8.0 Hz, 1H), 8.08 (dd, J=8.1, 1.5 Hz, 1H), 7.72 (s, 1H), 7.63 (t, J=8.0 Hz, 1H), 7.51 (s, 2H), 7.31 (d, J=8.8 Hz, 2H). ¹³C NMR (50 MHz, DMSO-d6): δ 172.12, 152.65 (2C), 148.26, 147.26, 137.49, 135.84, 131.80, 130.02, 128.63, 126.48, 121.94, 120.41, 119.84, 109.23. HPLC (λ₂₈₀): Purity 99.1%; t_{R}: 11.850 min (method 2).

4,5-dimethylthiazol-2-yl-N″-(4-bromoaniline-carbonyl)-guanidine (MTF429) Synthesized following the general procedure B using 1-(4,5-dimethylthiazol-2-yl) guanidine (1 g, 5.88 mmol) and 4-bromophenyl isocyanate (1.16 g, 5.88 mmol) to afford the title compound as a white powder (1.64 g, 76%). ¹H NMR (200 MHz, DMSO-d6): δ 9.68 (br. s, 2H), 8.34 (br. s, 2H), 7.47 (s, 4H), 2.20 (s, 3H), 2.13 (s, 3H). ¹³C NMR (50 MHz, DMSO-d6): δ 165.88, 154.26, 152.23, 142.31, 138.16, 131.57 (2C), 120.75 (2C), 117.16, 114.28, 14.46, 10.61. HPLC (λ₂₈₀): Purity 97.3%; t_{R}: 9.250 min (method 2).

N-4-(3-nitrophenyl) thiazol-2-yl-N″-(naphtalen-1-amine-carbonyl)-guanidine (MTF430) Synthesized following the general procedure B using 1-(4-(3-nitrophenyl) thiazol-2-yl) guanidine (1 g, 3.80 mmol) and 1-naphthyl isocyanate (0.55 mL, 3.80 mmol) to afford the title compound as an yellowish powder (1.27 g, 78%). ¹H NMR (400 MHz, DMSO-d6): δ 10.73 (s, 1H), 9.64 (s, 1H), 8.63 (s, 1H and br. s, 2H), 8.36 (d, J=7.8 Hz, 1H), 8.11 (dd, J=8.2, 1.2 Hz, 3H), 7.95 (d, J=8.0 Hz, 1H), 7.81 (s, 1H), 7.66 (dt, J=16.9, 7.3 Hz, 3H), 7.56 (t, J=7.3 Hz, 1H), 7.49 (t, J=7.9 Hz, 1H). ¹³C NMR (101 MHz, DMSO-d6): δ 172.64, 152.19, 152.11, 148.33, 147.64, 135.74, 133.67, 133.12, 132.03, 130.12, 128.60, 126.15, 126.10, 125.87, 125.54, 123.78, 122.09, 120.94, 119.87, 117.29, 109.15. HPLC (λ₂₈₀): Purity 98.7%; t_{R}: 12.492 min (method 2).

N-4-(3-nitrophenyl) thiazol-2-yl-N″-(3-methylaniline-carbonyl)-guanidine (MTF431) Synthesized following the general procedure B using 1-(4-(3-nitrophenyl) thiazol-2-yl) guanidine (1 g, 3.80 mmol) and m-tolyl isocyanate (0.50 mL, 3.80 mmol) to afford the title compound as an yellowish powder (732 mg, 47%). ¹H NMR (400 MHz, DMSO-d6): δ 9.39 (s, 2H), 8.63 (s, 1H), 8.55 (br. s, 2H), 8.34 (d, J=7.9 Hz, 1H), 8.12 (dd, J=8.1, 1.6 Hz, 1H), 7.77 (s, 1H), 7.67 (t, J=8.0 Hz, 1H), 7.28 (d, J=10.7 Hz, 2H), 7.18 (t, J=7.7 Hz, 1H), 6.85 (d, J=7.2 Hz, 1H), 2.28 (s, 3H). ¹³C NMR (101 MHz, DMSO-d6): δ 173.18, 152.34 (2C), 148.33, 147.44, 138.15 (2C), 135.88, 131.92, 130.14, 128.72, 123.74, 122.02, 119.85, 119.44, 116.11, 108.91, 21.16. HPLC (λ₂₈₀): Purity 100.0%; t_{R}: 11.400 min (method 2).

N-4-(3-nitrophenyl) thiazol-2-yl-N″-(3-methoxyaniline-carbonyl)-guanidine (MTF432) Synthesized following the general procedure B using 1-(4-(3-nitrophenyl) thiazol-2-yl) guanidine (1 g, 3.80 mmol) and 3-methoxyphenyl isocyanate (0.50 mL, 3.80 mmol) to afford the title compound as an yellowish powder (1.14 g, 74%). ¹H NMR (400 MHz, DMSO-d6): δ 9.60 (s, 1H), 9.38 (s, 1H), 8.82 (br. s, 2H), 8.65 (s, 1H) 8.36 (d, J=7.8 Hz, 1H), 8.16-8.11 (m, 1H), 7.81 (s, 1H), 7.70 (t, J=8.0 Hz, 1H), 7.20 (dd, J=18.0, 10.5 Hz, 2H), 6.96 (br. s, 1H), 6.62 (s, 1H), 3.74 (s, 3H). ¹³C NMR (101 MHz, DMSO-d6) o 173.20, 159.68, 152.30 (2C), 148.37, 147.55, 139.29, 135.87, 131.94, 130.21, 129.73, 122.07, 119.87, 111.22, 108.84, 108.50, 104.75, 55.00. HPLC (λ₂₈₀): Purity 100.0%; t_{R}: 11.058 min (method 2).

N-4-phenylthiazol-2-yl-N″-(2-methoxyaniline-carbonyl)-guanidine (MTF433) Synthesized following the general procedure B using 1-(4-phenylthiazol-2-yl) guanidine (1 g, 4.58 mmol) and 2-methoxyphenyl isocyanate (0.61 mL, 4.58 mmol) to afford the title compound as a white powder (470 mg, 28%). ¹H NMR (400 MHz, DMSO-d6): δ 10.88 (s, 1H), 9.46 (s, 1H), 9.13 (s, 1H), 8.17 (d, J=7.4 Hz, 1H), 7.93 (br. s and d, J=7.2 Hz, 3H), 7.52 (s, 1H), 7.42 (t, J=7.1 Hz, 2H), 7.36-7.28 (m, 1H), 7.04 (dt, J=15.2, 7.5 Hz, 2H), 6.92 (t, J=7.1 Hz, 1H), 3.95 (s, 3H). ¹³C NMR (50 MHz, DMSO-d6): δ 172.08, 151.65, 151.56, 149.84, 148.11, 134.25, 128.64 (2C), 127.68 (2C), 125.71 (2C), 122.98, 120.56, 118.70, 110.82, 106.57, 55.80. HPLC (λ₂₈₀): Purity 99.6%; t_{R}: 11.600 min (method 2).

N-4-(3-nitrophenyl) thiazol-2-yl-N″-(2-methoxyaniline-carbonyl)-guanidine (MTF434) Synthesized following the general procedure B using 1-(4-(3-nitrophenyl) thiazol-2-yl) guanidine (1 g, 3.80 mmol) and 2-methoxyphenyl isocyanate (0.51 mL, 3.80 mmol) to afford the title compound as an yellowish powder (978 mg, 63%). ¹H NMR (200 MHz, DMSO-d6): δ 10.80 (br. s, 1H), 9.52 (s, 1H), 8.65-8.59 (m, 1H), 8.35 (d, J=8.0 Hz, 1H), 8.27 (br. s, 2H), 8.22-8.14 (m, 1H), 8.10 (dd, J=7.8, 1.8 Hz, 1H), 7.78 (s, 1H), 7.65 (t, J=8.0 Hz, 1H), 7.09-6.83 (m, 3H), 3.93 (s, 3H). 13C NMR (50 MHz, DMSO-d6): δ 172.46, 151.73, 151.50, 148.33, 148.11, 147.55, 135.77, 131.99, 130.09, 127.62, 122.96, 122.05, 120.53, 119.83, 118.72, 110.74, 109.25, 55.77. HPLC (λ₂₈₀): Purity 99.0%; t_{R}: 11.783 min (method 2).

N-4-phenylthiazol-2-yl-N″-(naphtalen-1-amine-carbonyl)-guanidine (MTF435) Synthesized following the general procedure B using 1-(4-phenylthiazol-2-yl) guanidine (1 g, 4.58 mmol) and 1-naphthyl isocyanate (0.66 mL, 4.58 mmol) to afford the title compound as a white powder (1.43 g, 81%). ¹H NMR (200 MHz, DMSO-d6): δ 10.88 (br. s, 1H), 9.63 (s, 1H), 8.67 (br. s, 2H), 8.21-8.09 (m, 2H), 8.02-7.91 (m, 3H), 7.77-7.49 (m, 5H), 7.48-7.27 (m, 3H). ¹³C NMR (50 MHz, DMSO-d6): δ 172.24, 152.12 (2C), 149.94, 134.25, 133.69, 133.21, 128.65 (3C), 127.72, 126.18, 126.13, 125.91, 125.75 (2C), 125.60, 123.78, 120.99, 117.33, 106.46. HPLC (λ₂₈₀): Purity 97.0%; t_{R}: 12.358 min (method 2).

N-benzo[d]thiazol-2-yl-N″-(2-methylaniline-carbonyl)-guanidine (MTF495) Synthesized following the general procedure B using 1-(benzo[d]thiazol-2-yl) guanidine (0.5 g, 2.60 mmol) and o-tolyl isocyanate (0.32 mL, 2.60 mmol) to afford the title compound as an white powder (567 mg, 67%). ¹H NMR (400 MHz, DMSO-d6): δ 9.80 (s, 1H), 9.54 (s, 2H), 8.39 (s, 1H), 7.90 (d, J=7.6 Hz, 1H), 7.80 (d, J=7.8 Hz, 1H), 7.64 (d, J=7.3 Hz, 1H), 7.35 (t, J=7.3 Hz, 1H), 7.20 (dt, J=13.8, 4.7 Hz, 3H), 7.02 (t, J=7.3 Hz, 1H), 2.31 (s, 3H). ¹³C NMR (101 MHz, DMSO-d6): δ 171.99, 153.41, 152.09, 151.21, 136.15, 130.83, 130.34, 127.87, 126.36, 125.79, 123.75, 122.98, 121.25, 121.10, 119.70, 18.07. HPLC (λ₂₈₀): Purity 99.8%; t_{R}: 10.408 min (method 3).

4,5-dimethylthiazol-2-yl-N″-(aniline-carbonyl)-guanidine (MTF496) Synthesized following the general procedure B using 1-(4,5-dimethylthiazol-2-yl) guanidine (0.5 g, 2.94 mmol) and phenyl isocyanate (0.32 mL, 2.94 mmol) to afford the title compound as a white powder (705 mg, 83%). ¹H NMR (400 MHz, DMSO-d6): δ 9.63 (s, 1H), 9.25 (s, 1H), 8.30 (s, 2H), 7.46 (d, J=7.9 Hz, 2H), 7.31 (t, J=7.9 Hz, 2H), 7.03 (t, J=7.3 Hz, 1H), 2.19 (s, 3H), 2.13 (s, 3H). ¹³C NMR (101 MHz, DMSO-d6): δ 168.32, 153.06, 151.82, 142.46, 138.50, 128.90 (2C), 122.90, 118.88 (2C), 116.96, 14.51, 10.63. HPLC (λ₂₈₀): Purity 95.4%; t_{R}: 7.533 min (method 3).

N-benzo[d]thiazol-2-yl-N″-(2-chloroaniline-carbonyl)-guanidine (MTF504) Synthesized following the general procedure B using 1-(benzo[d]thiazol-2-yl) guanidine (0.5 g, 2.60 mmol) and 2-chlorophenyl isocyanate (0.31 mL, 2.60 mmol) to afford the title compound as an white powder (755 mg, 84%). ¹H NMR (400 MHz, DMSO-d6): δ 10.89 (s, 1H), 9.85 (s, 1H), 9.46 (s, 1H), 8.23 (d, J=8.0 Hz, 1H), 8.00 (s, 1H), 7.85 (d, J=7.7 Hz, 1H), 7.66 (d, J=8.0 Hz, 1H), 7.53 (dd, J=8.0, 1.2 Hz, 1H), 7.42-7.28 (m, 2H), 7.28-7.19 (m, 1H), 7.12 (td, J=8.0, 1.3 Hz, 1H). ¹³C NMR (101 MHz, DMSO-d6): δ 171.37, 152.95, 151.47, 150.97, 135.13, 131.03, 129.27, 127.73, 125.84, 124.30, 123.15, 122.54, 121.33 (2C), 119.87. HPLC (λ₂₈₀): Purity 98.1%; t_{R}: 11.383 min (method 3).

4,5-dimethylthiazol-2-yl-N″-(2-chloroaniline-carbonyl)-guanidine (MTF505) Synthesized following the general procedure B using 1-(4,5-dimethylthiazol-2-yl) guanidine (0.5 g, 2.94 mmol) and 2-chlorophenyl isocyanate (0.35 mL, 2.94 mmol) to afford the title compound as a white powder (638 mg, 67%). ¹H NMR (400 MHz, DMSO-d6): δ 11.55 (s, 1H), 9.42 (s, 1H), 9.09 (s, 1H), 8.27 (d, J=7.9 Hz, 1H), 7.48 (dd, J=8.0, 1.3 Hz, 1H), 7.41 (br. s, 1H), 7.37-7.24 (m, 1H), 7.06 (td, J=7.9, 1.4 Hz, 1H), 2.20 (s, 3H), 2.14 (s, 3H). ¹³C NMR (101 MHz, DMSO-d6) δ 167.62, 151.33, 150.95, 142.58, 135.49, 129.20, 127.71, 123.92, 122.13, 120.95, 117.80, 14.50, 10.60. HPLC (λ₂₈₀): Purity 99.5%; t_{R}: 10.350 min (method 3).

4,5-dimethylthiazol-2-yl-N″-(3-chloroaniline-carbonyl)-guanidine (MTF506) Synthesized following the general procedure B using 1-(4,5-dimethylthiazol-2-yl) guanidine (0.5 g, 2.94 mmol) and 3-chlorophenyl isocyanate (0.36 mL, 2.94 mmol) to afford the title compound as a white powder (676 mg, 71%). ¹H NMR (400 MHz, DMSO-d6): δ 9.67 (s, 2H), 8.33 (s, 2H), 7.73 (s, 1H), 7.39-7.26 (m, 2H), 7.09-7.01 (m, 1H), 2.19 (s, 3H), 2.13 (s, 3H). ¹³C NMR (101 MHz, DMSO-d6): δ 166.30, 154.85, 152.37, 142.25, 140.43, 133.24, 130.41, 122.29, 118.27, 117.28 (2C), 14.45, 10.60. HPLC (λ₂₈₀): Purity 95.4%; t_{R}: 8.833 min (method 3).

4,5-dimethylthiazol-2-yl-N″-(4-chloroaniline-carbonyl)-guanidine (MTF507) Synthesized following the general procedure F using 1-(4,5-dimethylthiazol-2-yl) guanidine (0.5 g, 2.94 mmol) and 4-chlorophenyl isocyanate (450 mg, 2.94 mmol) to afford the title compound as a white powder (866 mg, 91%). ¹H NMR (400 MHz, DMSO-d6): δ 9.67 (s, 1H), 9.47 (s, 1H), 8.32 (s, 2H), 7.51 (d, J=8.5 Hz, 2H), 7.33 (d, J=8.8 Hz, 2H), 2.19 (s, 3H), 2.12 (s, 3H). ¹³C NMR (101 MHz, DMSO-d6): δ 167.02, 153.88, 152.14, 142.32, 137.72, 128.68 (2C), 126.35, 120.40 (2C), 117.18, 14.45, 10.60. HPLC (λ₂₈₀): Purity 99.0%; t_{R}: 8.775 min (method 3).

4,5-dimethylthiazol-2-yl-N″-(2-methylaniline-carbonyl)-guanidine (MTF509) Synthesized following the general procedure B using 1-(4,5-dimethylthiazol-2-yl) guanidine (0.5 g, 2.94 mmol) and o-tolyl isocyanate (0.36 mL, 2.94 mmol) to afford the title compound as a white powder (687 mg, 77%). ¹H NMR (400 MHz, DMSO-d6): δ 10.24 (br. s, 1H), 9.32 (s, 1H), 8.99 (br. s, 1H), 7.96 (d and br. s, J=6.7 Hz, 2H), 7.17 (dd, J=16.6, 7.9 Hz, 2H), 6.97 (t, J=7.3 Hz, 1H), 2.29 (s, 3H), 2.19 (s, 3H), 2.13 (s, 3H). ¹³C NMR (101 MHz, DMSO-d6): δ 168.27, 151.81, 151.46, 142.63, 136.63, 130.23, 127.09, 126.32, 123.21, 120.38, 117.23, 18.25, 14.50, 10.60. HPLC (λ₂₈₀): Purity 97.8%; t_{R}: 7.695 min (method 3).

4,5-dimethylthiazol-2-yl-N″-(3-methylaniline-carbonyl)-guanidine (MTF510) Synthesized following the general procedure B using 1-(4,5-dimethylthiazol-2-yl) guanidine (0.5 g, 2.94 mmol) and m-tolyl isocyanate (0.38 mL, 2.94 mmol) to afford the title compound as a white powder (598 mg, 67%). ¹H NMR (400 MHz, DMSO-d6): δ 9.57 (s, 1H), 9.27 (s, 1H), 8.34 (s, 2H), 7.30 (s, 1H), 7.25 (d, J=8.2 Hz, 1H), 7.17 (t, J=7.7 Hz, 1H), 6.84 (d, J=7.4 Hz, 1H), 2.27 (s, 3H), 2.18 (s, 3H), 2.13 (s, 3H). ¹³C NMR (101 MHz, DMSO-d6): δ 167.96, 153.10, 151.90, 142.54, 138.42, 138.21, 128.77, 123.70, 119.42, 116.96, 116.08, 21.19, 14.53, 10.65. HPLC (λ₂₈₀): Purity 95.5%; t_{R}: 7.825 min (method 3).

4,5-dimethylthiazol-2-yl-N″-(3-methoxyaniline-carbonyl)-guanidine (MTF511) Synthesized following the general procedure B using 1-(4,5-dimethylthiazol-2-yl) guanidine (0.5 g, 2.94 mmol) and 3-methoxyphenyl isocyanate (0.39 mL, 2.94 mmol) to afford the title compound as a white powder (761 mg, 81%). ¹H NMR (400 MHz, DMSO-d6): δ 9.64 (s, 1H), 9.30 (s, 1H), 8.32 (br. s, 2H), 7.20 (dd, J=9.9, 6.3 Hz, 2H), 6.96 (d, J=7.4 Hz, 1H), 6.61 (dd, J=8.2, 2.1 Hz, 1H), 3.73 (s, 3H), 2.19 (s, 3H), 2.13 (s, 3H). ¹³C NMR (101 MHz, DMSO-d6): δ 167.77, 159.74, 153.33, 151.95, 142.46, 139.77, 129.68, 117.03, 111.14, 108.29, 104.65, 54.99, 14.49, 10.61. HPLC (λ₂₈₀): Purity 99.6%; t_{R}: 7.300 min (method 3).

4,5-dimethylthiazol-2-yl-N″-(3-nitroaniline-carbonyl)-guanidine (MTF512) Synthesized following the general procedure B using 1-(4,5-dimethylthiazol-2-yl) guanidine (0.5 g, 2.94 mmol) and 3-nitrophenyl isocyanate (483 mg, 2.94 mmol) to afford the title compound as a yellowish powder (629 mg, 64%). ¹H NMR (400 MHz, DMSO-d6): δ 9.79 (s, 2H), 8.62 (s, 1H), 8.31 (s, 2H), 7.83 (dd, J=8.0, 1.8 Hz, 2H), 7.55 (t, J=8.2 Hz, 1H), 2.20 (s, 3H), 2.12 (s, 3H). ¹³C NMR (101 MHz, DMSO-d6): δ 162.88, 156.40, 153.19, 148.05, 141.94, 140.61, 129.94, 124.79, 117.64, 116.77, 112.73, 14.36, 10.56. HPLC (λ₂₈₀): Purity 96.4%; t_{R}: 8.425 min (method 3).

N-4-(3-nitrophenyl) thiazol-2-yl-N″-(2-methylaniline-carbonyl)-guanidine (MTF515) Synthesized following the general procedure B using 1-(4-(3-nitrophenyl) thiazol-2-yl) guanidine (0.5 g, 1.90 mmol) and o-tolyl isocyanate (0.24 mL, 1.90 mmol) to afford the title compound as an yellowish powder (618 mg, 82%). ¹H NMR (400 MHz, DMSO) δ 9.88 (s, 1H), 9.53 (s, 1H), 8.60 (s and br. s, 3H), 8.33 (d, J=7.9 Hz, 1H), 8.10 (dd, J=8.1, 1.5 Hz, 1H), 7.94 (d, J=7.8 Hz, 1H), 7.75 (s, 1H), 7.65 (t, J=8.0 Hz, 1H), 7.17 (dd, J=14.3, 7.5 Hz, 2H), 6.98 (t, J=7.3 Hz, 1H), 2.29 (s, 3H). ¹³C NMR (101 MHz, DMSO-d6): δ 172.67, 152.05, 151.89, 148.32, 147.53, 136.40, 135.75, 131.99, 130.25, 130.11, 127.37, 126.32, 123.42, 122.05, 120.64, 119.83, 109.02, 18.16. HPLC (λ₂₈₀): Purity 98.4%; t_{R}: 10.608 min (method 3).

N-benzo[d]thiazol-2-yl-N″-(2,4-dichloroaniline-carbonyl)-guanidine (MTF649) 2-(Benzo[d]thiazol-2-yl) guanidine hydrochloride (912 mg, 4 mmol) was suspended in dioxane/DMF/CH₂Cl₂ (50 ml/5 mL/5 mL). 2,4-dichlorophenylisocyanate (752 mg, 4 mmol) was added, then DiPEA (700 μL, 4 mmol). A white precipitate appeared immediately. The reaction mixture was stirred for 16 h at room temperature, then the white suspension was poured onto water (175 mL) under vigorous stirring. The mixture was cooling down to 0° C., then filtered, water and the residue washed with cold and dried at air to afford the titled compound as white solid (1.10 g, 72%). ¹H NMR (200 MHz, DMSO-d6): δ 11.09 (br, 1H, NH), 9.86 (s, 1H, NH), 9.43 (br, 1H, NH), 8.25 (d, J=8.34 Hz, 1H, CHₐᵣ), 7.92 (br, 1H, NH), 7.83 (d, J=7.67 Hz, 1H, Chₐᵣ), 7.64 (m, 2H, CHₐᵣ), 7.39 (m, 2H, CHₐᵣ), 7.22 (t, J=7.35 Hz, 1H, CHₐᵣ) ¹³C NMR (50 MHz, DMSO-d₆): δ 171.15, 152.75, 151.26, 150.88, 134.35, 130.96, 128.62 (2C), 127.80, 127.19, 125.85, 123.19, 122.12, 121.34, 119.87 HPLC (λ²⁵⁴): Purity 99.5%; t_{R}: 6.22 min (method 4).

N-benzo[d]thiazol-2-yl-N″-(2,6-dichloroaniline-carbonyl)-guanidine (MTF650) 2-(Benzo[d]thiazol-2-yl) guanidine hydrochloride (912 mg, 4 mmol) was dissolved in dioxane/DMF/CH₂Cl₂ (50 mL/5 mL/5 mL). 2,6-dichlorophenylisocyanate (752 mg, 4 mmol) was added, then DiPEA (700 μL, 4 mmol). The colorless limpid solution was stirred for 16 h at room temperature, then the white suspension was poured onto water (175 mL) under vigorous stirring. The mixture was cooling down to 0° C., then filtered, water and the residue washed with cold and dried at air to afford the titled compound as white solid (1.21 g, 78%). ¹H NMR (200 MHz, DMSO-d₆): δ 10.14 (br, 1H, NH), 9.31 (br, 2H, NH), 8.55 (br, 1H, NH), 7.81 (d, J=7.4 Hz, 1H, CHₐᵣ), 7.59 (d, J=8.24 Hz, 3H, CHₐᵣ), 7.37 (m, 2H, CHₐᵣ), 7.18 (t, J=7.50 Hz, 1H, CHₐᵣ) ¹³C NMR (50 MHz, DMSO-d₆): δ 172.13, 153.27, 152.42, 151.09, 133.77, 131.78, 130.84, 129.23 (2C), 128.57 (2C), 125.71, 122.88, 121.19, 119.62. HRMS-ESI (m/z): [M+H]⁺ calc, for C₁₅H₁₂N₅OSCl₂⁺, 380.01341; Found: 380.01166. HPLC (λ²⁵⁴): Purity 98.4%; t_{R}: 11.52 min (HPLC analysis on a JASCO PU-2089/AS4050 apparatus with a Supelco analytical column Ascentis Express C18, 10 cm×4.6 mm, 5 μm, employing the following method: 5% B for 3 min, 50% B to 95% B over 12 min, 95% B for 5 min then from 95% B to 50% B over 2 min, 50% B for 3 min (total time: 25 min). Flow: 1 mL/min. Solvent A: water with 0.1% formic acid. Solvent B: acetonitrile with 0.1% formic acid).

N-benzo[d]thiazol-2-yl-N″-(2-bromoaniline-carbonyl)-guanidine (MTF651) 2-(Benzo[d]thiazol-2-yl) guanidine hydrochloride (456 mg, 2 mmol) was dissolved in dioxane (10 mL). DiPEA was added (350 μL, 2 mmol), then 2-bromophenylisocyanate (246 μL, 2 mmol). A white precipitate appeared immediately. The reaction mixture was stirred for 16 h at room temperature, then the white suspension was poured onto water (80 mL) under vigorous stirring. The mixture was cooling down to 0° C., then filtered, water and the residue washed with cold and dried at air to afford the titled compound as white solid (780 mg, 99%). ¹H NMR (200 MHz, DMSO-d₆): δ 10.58 (br, 1H, NH), 9.89 (br, 1H, NH), 9.49 (br, 1H, NH), 8.14 (d, J=7.85 Hz, 1H, CHₐᵣ), 8.00 (br, 1H, NH), 7.83 (d, J=7.99 Hz, 1H, CHₐᵣ), 7.65 (m, 2H, CHₐᵣ), 7.37 (m, 2H, CHₐᵣ), 7.22 (m, 1H, CHₐᵣ), 7.05 (m, 1H, CHₐᵣ) ¹³C NMR (50 MHz, DMSO-d₆): δ 171.50, 152.92, 151.57, 150.91, 136.21, 132.58, 130.99, 128.26, 125.87, 125.17, 123.17, 122.50, 121.36, 119.84, 113.45 HRMS-ESI (m/z): [M+H]⁺ calc. for C₁₅H₁₃N₅OSBr⁺, 391.99982; Found: 391.99798. HPLC (λ²⁵⁴): Purity 98.6%; t_{R}: 13.15 min (HPLC analysis on a JASCO PU-2089/AS4050 apparatus with a Supelco analytical column Ascentis Express C18, 10 cm×4.6 mm, 5 μm, employing the following method: 5% B for 3 min, 50% B to 95% B over 12 min, 95% B for 5 min then from 95% B to 50% B over 2 min, 50% B for 3 min (total time: 25 min). Flow: 1 mL/min. Solvent A: water with 0.1% formic acid. Solvent B: acetonitrile with 0.1% formic acid).

N-benzo[d]thiazol-2-yl-N″-(2-ethoxyaniline-carbonyl)-guanidine (MTF652) 2-(Benzo[d]thiazol-2-yl) guanidine hydrochloride (456 mg, 2 mmol) was dissolved in dioxane (16 mL). DiPEA was added (350 μL, 2 mmol), then 2-ethoxyphenylisocyanate (303 μL, 2 mmol). A white precipitate appeared immediately. The reaction mixture was stirred for 16 h at room temperature, then the white suspension was poured onto water (80 mL) under vigorous stirring. The mixture was cooling down to 0° C., then filtered, water and the residue washed with cold and dried at air to afford the titled compound as white solid (622 mg, 88%). ¹H NMR (200 MHz, DMSO-d₆): δ 10.14 (br, 1H, NH), 9.46 (br, 2H, NH), 8.55 (br, 1H, NH), 8.07 (d, J=7.6 Hz, 1H, CHₐᵣ), 7.80 (d, J=7.7 Hz, 1H, CHₐᵣ), 7.62 (d, J=7.8 Hz, 1H, CHₐᵣ), 7.36 (t, J=7.5 Hz, 1H, CHₐᵣ), 7.21 (t, J=7.5 Hz, 1H, CHₐᵣ), 7.03 (m, 2H, CHₐᵣ), 6.90 (t, J=7.1 Hz, 1H, CHₐᵣ), 4.17 (q, J=6.7 Hz, 2H, CH₂), 1.41 (t, J=6.7 Hz, 3H, CH₃). ¹³C NMR (50 MHz, DMSO-d₆): δ 172.20, 153.37, 152.12, 151.28, 147.34, 130.79, 127.43, 125.74, 123.31, 122.88, 121.22, 120.44, 119.58, 119.17, 111.99, 64.04, 14.62. HRMS-ESI (m/z): [M+H]⁺ calc, for C₁₇H₁₈N₅O₂S⁺, 356.11757; Found: 356.11613. HPLC (λ₂₅₄): Purity 99.6%; t_{R}: 4.97 min (HPLC analysis on a JASCO PU-2089/AS4050 apparatus with a Supelco analytical column Ascentis Express C18, 10 cm×4.6 mm, 5 μm, employing the following method: 50% B for 3 min, 50% B to 95% B over 2 min, 95% B for 10 min then from 95% B to 50% B over 2 min, 50% B for 3 min (total time: 20 min). Flow: 1 mL/min. Solvent A: water with 0.1% formic acid. Solvent B: acetonitrile with 0.1% formic acid).

N-benzo[d]thiazol-2-yl-N″-(2-ethylaniline-carbonyl)-guanidine (MTF653) 2-(Benzo[d]thiazol-2-yl) guanidine hydrochloride (456 mg, 2 mmol) was dissolved in dioxane (10 mL). DiPEA was added (350 μL, 2 mmol), then 2-bromophenylisocyanate (282 μL, 2 mmol). A white precipitate appeared immediately. The reaction mixture was stirred for 16 h at room temperature, then the white suspension was poured onto water (80 mL) under vigorous stirring. The mixture was cooling down to 0° C., then filtered, water and the residue washed with cold and dried at air to afford the titled compound as white solid (678 mg, 99%). ¹H NMR (200 MHz, DMSO-d₆): δ 9.79 (br, 1H, NH), 9.50 (br, 2H, NH), 8.41 (br, 1H, NH), 7.82 (m, 2H, CHₐᵣ), 7.62 (m, 1H, CHₐᵣ), 7.36 (t, J=7.36 Hz, 1H, CHₐᵣ), 7.21 (m, 3H, Chₐᵣ), 7.07 (t, J=7.0 Hz, 1H, CHₐᵣ), 2.67 (q, J=7.4 Hz, 2H, CH₂), 1.21 (t, J=7.4 Hz, 3H, CH₃). ¹³C NMR (50 MHz, DMSO-d₆): δ 171.97, 153.37, 152.25, 151.18, 135.22, 134.01, 130.78, 128.57, 126.27, 125.79, 124.24, 122.97, 121.98, 121.26, 119.67, 66.34, 14.16. HPLC (λ₂₅₄): Purity 99.9+%; t_{R}: 5.05 min (HPLC analysis on a JASCO PU-2089/AS4050 apparatus with a Supelco analytical column Ascentis Express C18, 10 cm×4.6 mm, 5 μm, employing the following method: 50% B for 3 min, 50% B to 95% B over 2 min, 95% B for 10 min then from 95% B to 50% B over 2 min, 50% B for 3 min (total time: 20 min). Flow: 1 mL/min. Solvent A: water with 0.1% formic acid. Solvent B: acetonitrile with 0.1% formic acid).

N-benzo[d]thiazol-2-yl-N″-(2-fluoroaniline-carbonyl)-guanidine (MTF654) 2-(Benzo[d]thiazol-2-yl) guanidine hydrochloride (456 mg, 2 mmol) was dissolved in dioxane (10 mL). DiPEA was added (350 μL, 2 mmol), then 2-fluorophenylisocyanate (224 μL, 2 mmol). A white precipitate appeared immediately. The reaction mixture was stirred for 16 h at room temperature, then the white suspension was poured onto water (80 mL) under vigorous stirring. The mixture was cooling down to 0° C., then filtered, water and the residue washed with cold and dried at air to afford the titled compound as white solid (566 mg, 86%). ¹H NMR (200 MHz, DMSO-d₆): δ 10.34 (br, 1H, NH), 9.79 (br, 1H, NH), 9.42 (br, 1H, NH), 8.31 (br, 1H, NH), 8.12 (s, 1H, CHₐᵣ), 7.83 (s, 1H, CHₐᵣ), 7.63 (s, 1H, CHₐᵣ), 7.21 (m, 5H, CHₐᵣ). ¹³C NMR (50 MHz, DMSO-d₆): δ 171.69, 153.61, 153.02, 151.78, 151.19, 130.93, 126.15 (J_{CF}=10.59 Hz), 125.79, 124.67 (J_{CF}=3.08 Hz), 123.94 (J_{CF}=6.25 Hz), 123.03, 121.29, 121.04, 119.73, 115.19 (J_{CF}=19.13 Hz). ¹⁹F NMR (376 MHz, DMSO-d₆) δ −128.94. HPLC (λ₂₅₄): Purity 99.6%; t_{R}: 4.70 min (HPLC analysis on a JASCO PU-2089/AS4050 apparatus with a Supelco analytical column Ascentis Express C18, 10 cm×4.6 mm, 5 μm, employing the following method: 50% B for 3 min, 50% B to 95% B over 2 min, 95% B for 10 min then from 95% B to 50% B over 2 min, 50% B for 3 min (total time: 20 min). Flow: 1 mL/min. Solvent A: water with 0.1% formic acid. Solvent B: acetonitrile with 0.1% formic acid).

N-benzo[d]thiazol-2-yl-N″-(2-nitroaniline-carbonyl)-guanidine (MTF655) 2-(Benzo[d]thiazol-2-yl) guanidine hydrochloride (456 mg, 2 mmol) was dissolved in dioxane (10 mL). DiPEA was added (350 μL, 2 mmol), then 2-nitrophenylisocyanate (246 μL, 2 mmol). The yellow reaction mixture was stirred for 16 h at room temperature, then the white suspension was poured onto water (80 mL) under vigorous stirring. The mixture was cooling down to 0° C., then filtered, water and the residue washed with cold and dried at air to afford the titled compound as bright yellow solid (594 mg, 83%). ¹H NMR (200 MHz, DMSO-d₆): δ 10.99 (br, 1H, NH), 10.33 (br, 1H, NH), 9.44 (br, 1H, NH), 8.31 (br, 1H, NH), 8.24 (d, J=7.96 Hz, 1H, CHₐᵣ), 8.10 (dd, J³=8.38 Hz J⁴=1.20 Hz, 1H, CHₐᵣ), 7.85 (d, J=7.90 Hz, 1H, CHₐᵣ), 7.74 (t, J=7.91 Hz, 1H, CHₐᵣ), 7.65 (d, J=8.08 Hz, 1H, CHₐᵣ), 7.36 (t, J=7.57 Hz, 1H, CHₐᵣ), 7.30 (t, J=7.57 Hz, 1H, CHₐᵣ), 7.22 (t, J=7.74 Hz, 1H, CHₐᵣ). ¹³C NMR (50 MHz, DMSO-d₆): δ 171.96, 153.04, 150.78, 138.98, 134.80, 133.03, 131.15 (2C), 125.78, 125.36, 123.70, 123.07, 121.27, 119.73 (2C). HRMS-ESI (m/z): [M+H]⁺ calc. for C₁₅H₁₃N₆O₃S⁺, 357.07644; Found: 357.07477. HPLC (>254): Purity 95.2%; t_{R}: 11.76 min (HPLC analysis on a JASCO PU-2089/AS4050 apparatus with a Supelco analytical column Ascentis Express C18, 10 cm×4.6 mm, 5 μm, employing the following method: 5% B for 3 min, 50% B to 95% B over 12 min, 95% B for 5 min then from 95% B to 50% B over 2 min, 50% B for 3 min (total time: 25 min). Flow: 1 mL/min. Solvent A: water with 0.1% formic acid. Solvent B: acetonitrile with 0.1% formic acid).

N-benzo[d]thiazol-2-yl-N″-(2-methyl-5-chloroaniline-carbonyl)-guanidine (MTF656) 2-(Benzo[d]thiazol-2-yl) guanidine hydrochloride (456 mg, 2 mmol) was dissolved in dioxane (10 mL). DiPEA was added (350 μL, 2 mmol), then 5-chloro-2-methylbromophenylisocyanate (274 μL, 2 mmol). A white precipitate appeared immediately. The reaction mixture was stirred for 16 h at room temperature, then the white suspension was poured onto water (80 mL) under vigorous stirring. The mixture was cooling down to 0° C., then filtered, water and the residue washed with cold and dried at air to afford the titled compound as white solid (716 mg, 99%). ¹H NMR (200 MHz, DMSO-d₆): δ 9.85 (br, 2H, NH), 9.48 (br, 1H, NH), 8.27 (br, 1H, NH), 8.08 (s, 1H, Chₐᵣ), 7.82 (d, J=7.80 Hz, 1H, CHₐᵣ), 7.64 (d, J=7.89 Hz, 1H, CHₐᵣ), 7.36 (t, J=7.60 Hz, 1H, CHₐᵣ), 7.23 (m, 2H, CHₐᵣ), 7.06 (dd, J=8.06 Hz, J=2.09 Hz, 1H, CHₐᵣ), 2.30 (s, 3H, CH₃). ¹³C NMR (50 MHz, DMSO-d₆): δ 171.70, 153.15, 151.84, 151.09, 137.68, 131.69, 130.82, 130.50 (2C), 125.88, 123.12, 122.93, 121.32, 119.78, 119.64, 17.65. HRMS-ESI (m/z): [M+H]⁺ calc. for C₁₆H₁₅N₅OSCl⁺, 360.06803; Found: 360.06642. HPLC (>254): Purity 92.7%; t_{R}: 13.57 min (HPLC analysis on a JASCO PU-2089/AS4050 apparatus with a Supelco analytical column Ascentis Express C18, 10 cm×4.6 mm, 5 μm, employing the following method: 5% B for 3 min, 50% B to 95% B over 12 min, 95% B for 5 min then from 95% B to 50% B over 2 min, 50% B for 3 min (total time: 25 min). Flow: 1 mL/min. Solvent A: water with 0.1% formic acid. Solvent B: acetonitrile with 0.1% formic acid).

N-6-nitrobenzo[d]thiazol-2-yl-N″-(2-chloroaniline-carbonyl)-guanidine (MTF657) 2-(6-nitrobenzo[d]thiazol-2-yl) guanidine (237 mg, 1 mmol) was dissolved in dioxane (10 mL). Then 2-chlorophenylisocyanate (121 μL, 1 mmol) was added. The light red solution was stirred for 16 h at room temperature, then the milky lila suspension was poured onto water (80 mL) under vigorous stirring, and turned immediately fluorescent green upon contact with water. The mixture was cooling down to 0° C., then filtered, water and the residue washed with cold and dried at air to afford the titled compound as green solid (339 mg, 87%). ¹H NMR (200 MHz, DMSO-d₆): δ 11.69 (br, 1H, NH), 11.38 (br, 1H, NH), 8.87 (br, 1H, NH), 7.88 (br, 2H, NH+CHₐᵣ), 7.51 (m, 2H, CHₐᵣ), 7.35 (m, 2H, CHₐᵣ), 7.18 (m, 2H, CHₐᵣ). ¹³C NMR (50 MHz, DMSO-d₆): δ 159.90, 157.70, 152.44, 144.64, 134.89, 129.42, 129.37, 129.09, 128.94, 127.63, 127.40, 125.78, 124.03, 116.78, 115.42. HPLC (λ₂₅₄): Purity 99.4%; t_{R}: 4.89 min (HPLC analysis on a JASCO PU-2089/AS4050 apparatus with a Supelco analytical column Ascentis Express C18, 10 cm×4.6 mm, 5 μm, employing the following method: 50% B for 3 min, 50% B to 95% B over 2 min, 95% B for 10 min then from 95% B to 50% B over 2 min, 50% B for 3 min (total time: 20 min). Flow: 1 mL/min. Solvent A: water with 0.1% formic acid. Solvent B: acetonitrile with 0.1% formic acid).

MTF686: N-benzo[d]thiazol-2-yl-N″-(3,4,5-trimethoxyaniline-carbonyl)-guanidine. A mixture of 3,4,5-trimethoxyaniline (500 mg, 2.73 mmol), NMM (360 μL, 3.27 mmol) in dry THF (10 mL) was added in one portion at −55° C. to a solution of trisphogene (283.4 mg, 0.9552 mmol) in dry THF (5 mL). After addition, benzothiazolylguanidine (472.2 mg, 2.46 mmol) in dry DMF (8 mL) was added in one portion to the mixture. The mixture was stirred overnight at rt. Then, the mixture was cooled down to 0° C., precipitated and filtered. The filtrate was evaporated to remove the THF, the mixture remaining was poured into cold water and the solid was collected upon filtration and dried with pentane. The solid was then purified by silica gel column chromatography (cyclohexane/acetone, 10/0 to 6/4) to afford the pure desired product as white powder (111 mg, 10%). ¹H NMR (400 MHz, DMSO-d₆): δ 9.68 (s, 1H), 9.39 (s, 2H), 8.69 (s, 1H), 7.83-7.78 (m, 1H), 7.64 (d, J=8.1 Hz, 1H), 7.41-7.31 (m, 1H), 7.21 (td, J=7.6, 1.2 Hz, 1H), 6.82 (s, 2H), 3.77 (s, 6H), 3.64 (s, 3H). ¹³C NMR (101 MHz, DMSO-d₆): δ 174.29, 152.96, 152.91, 152.40 (2C), 150.70, 133.65, 132.96, 130.29, 125.21, 122.33, 120.67, 119.03, 96.44 (2C), 59.59, 55.25 (2C). HPLC (λ²⁵⁴): Purity 95.3%; t_{R}: 8.42 min (HPLC analysis on a JASCO PU-2089/AS4050 apparatus with a Supelco analytical column Ascentis Express C18, 10 cm×4.6 mm, 5 μm, employing the following method: 5% B for 2 min, 5% B to 95% B over 9 min, 95% B for 5 min then from 95% B to 5% B over 1 min, 5% B for 3 min (total time: 20 min). Flow: 1 mL/min. Solvent A: water with 0.1% formic acid. Solvent B: acetonitrile with 0.1% formic acid).

MTF687: N-benzo[d]thiazol-2-yl-N″-(3,4-dimethoxyaniline-carbonyl)-guanidine. A mixture of 3,4-dimethoxyaniline (500 mg, 3.26 mmol), NMM (431 μL, 3.92 mmol) in dry THF (10 mL) was added in one portion at −55° C. to a solution of trisphogene (339.0 mg, 1.14 mmol) in dry THF (5 mL). After addition, benzothiazolylguanidine (564.7 mg, 2.94 mmol) in dry DMF (8 mL) was added in one portion to the mixture. The mixture was stirred overnight at rt. Then, the mixture was cooled down to 0° C., precipitated and filtered. The filtrate was evaporated to remove the THF, the mixture remaining was poured into cold water and the solid was collected upon filtration and dried with pentane. The solid was then purified by silica gel column chromatography (cyclohexane/acetone, 10/0 to 6/4) to afford the pure desired product as white powder (229 mg, 19%). ¹H NMR (400 MHz, DMSO-d₆): δ 9.65 (s, 1H), 9.34 (d, J=30.2 Hz, 2H), 8.71 (s, 1H), 7.80 (d, J=7.8 Hz, 1H), 7.62 (s, 1H), 7.35 (t, J=7.6 Hz, 1H), 7.19 (dd, J=15.2, 7.4 Hz, 2H), 6.93 (d, J=9.0 Hz, 2H), 3.76 (s, 3H), 3.73 (s, 3H). ¹³C NMR (101 MHz, DMSO-d₆): δ 172.63, 153.70, 152.65, 151.51, 149.02, 145.28, 131.59, 130.98, 125.92, 123.02, 121.39, 119.72, 112.49, 111.45, 104.81, 55.97, 55.65. HPLC (λ²⁵⁴): Purity 100%; t_{R}: 8.183 min (HPLC analysis on a JASCO PU-2089/AS4050 apparatus with a Supelco analytical column Ascentis Express C18, 10 cm×4.6 mm, 5 μm, employing the following method: 5% B for 2 min, 5% B to 95% B over 9 min, 95% B for 5 min then from 95% B to 5% B over 1 min, 5% B for 3 min (total time: 20 min). Flow: 1 mL/min. Solvent A: water with 0.1% formic acid. Solvent B: acetonitrile with 0.1% formic acid).

MTF688: N-benzo[d]thiazol-2-yl-N″-(4-nitroaniline-carbonyl)-guanidine. In a 50 mL round-bottom flask, 1-(benzo[d]thiazol-2-yl) guanidine (500 mg, 2.60 mmol) was added to dioxane (25 mL) at room temperature. Once the compound had completely dissolved, 4-nitrophenyl isocyanate (427 mg, 2.60 mmol) was introduced in one portion, leading to the progressive formation of a white precipitate. After completion of the reaction (TLC monitoring), the precipitate was filtered, then washed twice with technical grade ethanol (about 20 mL) to afford the titled compound as a white powder (927 mg, 77%). ¹H NMR (500 MHz, DMSO-d₆): δ 9.97 (s, 2H), 9.37 (s, 1H), 8.60 (s, 1H), 8.23 (d, J=8.7 Hz, 2H), 7.94-7.71 (m, 3H), 7.66 (d, J=8.1 Hz, 1H), 7.38 (t, J=7.7 Hz, 1H), 7.24 (t, J=7.5 Hz, 1H). ¹³C NMR (101 MHz, DMSO-d₆): δ 170.88, 158.79, 153.02, 146.38, 141.42, 132.88, 130.66, 125.38, 124.56 (2C), 122.58, 120.77, 119.25, 117.95 (2C). HPLC (λ₂₅₄): Purity: 96.5%, t_{R}: 9.060 min (HPLC analysis on a JASCO PU-2089/AS4050 apparatus with a Supelco analytical column Ascentis Express C18, 10 cm×4.6 mm, 5 μm, employing the following method: 5% B for 2 min, 5% B to 95% B over 9 min, 95% B for 5 min then from 95% B to 5% B over 1 min, 5% B for 3 min (total time: 20 min). Flow: 1 mL/min. Solvent A: water with 0.1% formic acid. Solvent B: acetonitrile with 0.1% formic acid).

MTF689: N-benzo[d]thiazol-2-yl-N″-(2,6-dimethylaniline-carbonyl)-guanidine. In a 50 mL round-bottom flask, 1-(benzo[d]thiazol-2-yl) guanidine (237 mg, 995 μmol) was added to dioxane (25 mL). Once the compound had completely dissolved, 2-methoxyphenyl isocyanate (148 mg, 132 μl, 995 μmol) was introduced in one portion, leading to the progressive formation of a white precipitate. After completion of the reaction (TLC monitoring), the precipitate was filtered, then washed twice with technical grade ethanol (about 20 mL) to afford the titled compound as a white powder (320 mg, 83%). ¹H NMR (400 MHz, DMSO-d₆): δ 9.86 (s, 1H), 9.35 (s, 1H), 8.75 (s, 1H), 8.52 (s, 1H), 7.80 (d, J=7.9 Hz, 1H), 7.64 (d, J=8.1 Hz, 1H), 7.36 (t, J=7.7 Hz, 1H), 7.20 (t, J=7.6 Hz, 1H), 7.11 (s, 3H), 2.23 (s, 6H). ¹³C NMR (101 MHz, DMSO-d₆): δ 171.92, 153.18, 152.25, 150.86, 134.75 (2C), 133.24, 130.28, 127.35 (2C), 126.14, 125.18, 122.28, 120.65, 119.04, 17.60 (2C). HPLC (λ254): Purity 99.0%, t_{R}: 8.753 min (HPLC analysis on a JASCO PU-2089/AS4050 apparatus with a Supelco analytical column Ascentis Express C18, 10 cm×4.6 mm, 5 μm, employing the following method: 5% B for 2 min, 5% B to 95% B over 9 min, 95% B for 5 min then from 95% B to 5% B over 1 min, 5% B for 3 min (total time: 20 min). Flow: 1 mL/min. Solvent A: water with 0.1% formic acid. Solvent B: acetonitrile with 0.1% formic acid).

MTF690: N-benzo[d]thiazol-2-yl-N″-(3-methoxyaniline-carbonyl)-guanidine. In a 50 mL round-bottom flask, 1-(benzo[d]thiazol-2-yl) guanidine (500 mg, 2.60 mmol) was added to dioxane (25 mL) at room temperature. Once the compound had completely dissolved, 1-isocyanato-3-methoxybenzene (388 mg, 2.60 mmol) was introduced in one portion, leading to the progressive formation of a white precipitate. After completion of the reaction (TLC monitoring), the precipitate was filtered, then washed twice with technical grade ethanol (about 20 mL) to afford the titled compound as a white powder (716.5 mg, 81%). ¹H NMR (500 MHz, DMSO-d₆): δ 9.66 (s, 1H), 9.52 (s, 1H), 9.27 (s, 1H), 8.66 (s, 1H), 7.81 (d, J=7.8 Hz, 1H), 7.64 (d, J=8.0 Hz, 1H), 7.42-7.32 (m, 1H), 7.22 (td, J=15.4, 14.7, 7.4 Hz, 3H), 6.97 (s, 1H), 6.66 (dd, J=8.5, 2.5 Hz, 1H), 3.75 (s, 3H). ¹³C NMR (101 MHz, DMSO-d₆): δ 159.24, 152.96, 152.09, 150.67, 138.84, 130.37, 129.25, 125.22, 122.35, 120.68, 119.09, 117.84, 110.80, 108.16, 104.32, 54.51. HPLC (λ₂₅₄): Purity 95.6%; t_{R}: 8.963 min (HPLC analysis on a JASCO PU-2089/AS4050 apparatus with a Supelco analytical column Ascentis Express C18, 10 cm×4.6 mm, 5 μm, employing the following method: 5% B for 2 min, 5% B to 95% B over 9 min, 95% B for 5 min then from 95% B to 5% B over 1 min, 5% B for 3 min (total time: 20 min). Flow: 1 mL/min. Solvent A: water with 0.1% formic acid. Solvent B: acetonitrile with 0.1% formic acid).

MTF691: N-benzo[d]thiazol-2-yl-N″-(3-iodoaniline-carbonyl)-guanidine. In a 50 mL round-bottom flask, 1-(benzo[d]thiazol-2-yl) guanidine (500 mg, 2.60 mmol) was added to dioxane (25 mL). Once the compound had completely dissolved, 1-iodo-3-isocyanatobenzene (637 mg, 343 μL, 2.60 mmol) was introduced in one portion, leading to the progressive formation of a white precipitate. After completion of the reaction (TLC monitoring), the precipitate was filtered, then washed twice with technical grade ethanol (about 20 mL) to afford the titled compound as a white powder (78.6 mg, 6.9%). ¹H NMR (400 MHz, DMSO-d₆): δ 9.68 (d, J=87.7 Hz, 2H), 9.24 (s, 1H), 8.64 (s, 1H), 8.03 (s, 1H), 7.82 (dd, J=8.0, 1.3 Hz, 1H), 7.65 (d, J=8.0 Hz, 1H), 7.46-7.34 (m, 2H), 7.33 (s, 1H), 7.22 (td, J=7.6, 1.2 Hz, 1H), 7.14 (t, J=8.0 Hz, 1H). ¹³C NMR (101 MHz, DMSO-d₆): δ 171.20, 153.00, 150.51, 139.26, 131.16, 130.52, 130.42, 126.68, 25.30, 122.45, 120.73 (2C), 119.15, 117.88, 94.24. HPLC (λ₂₅₄): Purity 98,002%, t_{R}: 10.307 min (HPLC analysis on a JASCO PU-2089/AS4050 apparatus with a Supelco analytical column Ascentis Express C₁₈, 10 cm×4.6 mm, 5 μm, employing the following method: 5% B for 2 min, 5% B to 95% B over 9 min, 95% B for 5 min then from 95% B to 5% B over 1 min, 5% B for 3 min (total time: 20 min). Flow: 1 mL/min. Solvent A: water with 0.1% formic acid. Solvent B: acetonitrile with 0.1% formic acid).

MTF692: N-benzo[d]thiazol-2-yl-N″-(3-fluoroaniline-carbonyl)-guanidine. In a 50 mL round-bottom flask, 1-(benzo[d]thiazol-2-yl) guanidine (500 mg, 2.60 mmol) was added to dioxane (25 mL). Once the compound had completely dissolved, 3-fluorophenyl isocyanate (357 mg, 297 μl, 2.60 mmol) was introduced in one portion, leading to the progressive formation of a white precipitate. After completion of the reaction (TLC monitoring), the precipitate was filtered, then washed twice with technical grade ethanol (about 20 mL) to afford the titled compound as a white powder (389.8 mg, 46%). ¹H NMR (400 MHz, DMSO-d₆): δ 9.72 (s, 2H), 9.41 (s, 1H), 8.63 (s, 1H), 7.82 (d, J=7.8 Hz, 1H), 7.65 (d, J=8.0 Hz, 1H), 7.51 (d, J=11.5 Hz, 1H), 7.42-7.30 (m, 2H), 7.28-7.19 (m, 1H), 7.20-7.07 (s, 1H), 6.90 (t, J=8.8 Hz, 1H). ¹³C NMR (101 MHz, DMSO-d₆): δ 171.23, 161.80 (d, J=242 Hz), 156.60, 152.97, 150.48, 139.61, 130.44, 129.98 (d, J=9.09 Hz), 125.24, 122.40, 120.68, 119.12, 114.28, 108.99 (d, J=21.21 Hz), 105.28 (d, J=26.26 Hz). ¹⁹F NMR (376 MHz, DMSO-d₆): δ −111.88. HPLC (λ₂₅₄): Purity 98.6%; t_{R}: 9.240 min (HPLC analysis on a JASCO PU-2089/AS4050 apparatus with a Supelco analytical column Ascentis Express C18, 10 cm×4.6 mm, 5 μm, employing the following method: 5% B for 2 min, 5% B to 95% B over 9 min, 95% B for 5 min then from 95% B to 5% B over 1 min, 5% B for 3 min (total time: 20 min). Flow: 1 mL/min. Solvent A: water with 0.1% formic acid. Solvent B: acetonitrile with 0.1% formic acid).

MTF693: N-benzo[d]thiazol-2-yl-N″-(cylohexylcarbonyl)-guanidine. In a 50 mL round-bottom flask, 1-(benzo[d]thiazol-2-yl) guanidine (500 mg, 2.60 mmol) was added to dioxane (25 mL). Once the compound had completely dissolved, isocyanatocyclohexane (326 mg, 332 μl, 2.60 mmol) was introduced in one portion, leading to the progressive formation of a white precipitate. After completion of the reaction (TLC monitoring), the precipitate was filtered, then washed twice with technical grade ethanol (about 20 mL) to afford the titled compound as a white powder (312 mg, 38%). ¹H NMR (500 MHz, DMSO-d₆): δ 9.41 (s, 1H), 9.30 (s, 1H), 8.80 (s, 1H), 7.82 (dd, J=7.9, 1.2 Hz, 1H), 7.65 (d, J=7.7 Hz, 1H), 7.38 (td, J=7.7, 1.3 Hz, 1H), 7.23 (td, J=7.6, 1.2 Hz, 2H), 3.58 (dd, J=8.3, 4.3 Hz, 1H), 1.85 (dq, J=12.7, 3.9 Hz, 2H), 1.70 (dp, J=13.0, 4.4 Hz, 2H), 1.57 (dq, J=13.4, 4.6 Hz, 1H), 1.39 (tt, J=10.4, 3.2 Hz, 2H), 1.36-1.22 (m, 3H). ¹³C NMR (101 MHz, DMSO-d₆): δ 172.07, 153.40, 153.33, 150.93, 130.18, 125.13, 122.16, 120.60, 118.91, 47.12, 31.81 (2C), 24.60, 23.48 (2C). HPLC (λ₂₅₄): Purity: 99,018% t_{R}: 8.793 min (HPLC analysis on a JASCO PU-2089/AS4050 apparatus with a Supelco analytical column Ascentis Express C18, 10 cm×4.6 mm, 5 μm, employing the following method: 5% B for 2 min, 5% B to 95% B over 9 min, 95% B for 5 min then from 95% B to 5% B over 1 min, 5% B for 3 min (total time: 20 min). Flow: 1 mL/min. Solvent A: water with 0.1% formic acid. Solvent B: acetonitrile with 0.1% formic acid).

MTF694: N-benzo[d]thiazol-2-yl-N″-(3-chloro-4-methylaniline-carbonyl)-guanidine. In a 50 mL round-bottom flask, 1-(benzo[d]thiazol-2-yl) guanidine (500 mg, 2.60 mmol) was added to dioxane (25 mL). Once the compound had completely dissolved, 3-Chloro-4-methylphenyl isocyanate (434 mg, 354 μl, 2.60 mmol) was introduced in one portion, leading to the progressive formation of a white precipitate. After completion of the reaction (TLC monitoring), the precipitate was filtered, then washed twice with technical grade ethanol (about 20 mL) to afford the titled compound as a white powder (814.3 mg, 99%). ¹H NMR (500 MHz, DMSO-d₆): δ 9.65 (d, J=114.4 Hz, 2H), 9.22 (s, 1H), 8.71 (d, J=68.8 Hz, 1H), 7.81 (d, J=7.8 Hz, 1H), 7.70 (s, 1H), 7.64 (d, J=8.1 Hz, 1H), 7.36 (t, J=7.7 Hz, 1H), 7.29 (d, J=7.9 Hz, 1H), 7.22 (q, J=8.9, 7.6 Hz, 2H), 2.28 (s, 3H). ¹³C NMR (101 MHz, DMSO-d₆): δ 170.93, 153.01, 152.13, 150.53, 136.88, 132.74, 130.75, 130.45, 129.25, 125.23, 122.38, 120.67, 119.12, 118.53, 117.25, 18.36. HPLC (λ₂₅₄): Purity: 95.3%; t_{R}: 10.397 min (HPLC analysis on a JASCO PU-2089/AS4050 apparatus with a Supelco analytical column Ascentis Express C18, 10 cm×4.6 mm, 5 μm, employing the following method: 5% B for 2 min, 5% B to 95% B over 9 min, 95% B for 5 min then from 95% B to 5% B over 1 min, 5% B for 3 min (total time: 20 min). Flow: 1 mL/min. Solvent A: water with 0.1% formic acid. Solvent B: acetonitrile with 0.1% formic acid).

MTF695: N-benzo[d]thiazol-2-yl-N″-(4-fluoroaniline-carbonyl)-guanidine. In a 50 mL round-bottom flask, 1-(benzo[d]thiazol-2-yl) guanidine (500 mg, 2.60 mmol) was added to dioxane (25 mL) at room temperature. Once the compound had completely dissolved, 4-fluorophenylisocyanate (350 mg, 290 μL, 2.60 mmol) was introduced in one portion, leading to the progressive formation of a white precipitate. After completion of the reaction (TLC monitoring), the precipitate was filtered, then washed twice with technical grade ethanol (about 20 mL) to afford the titled compound as a white powder (508.8 mg, 60%). ¹H NMR (400 MHz, DMSO-d₆): δ 9.73 (s, 1H), 9.51 (s, 1H), 9.29 (s, 1H), 8.66 (s, 1H), 7.81 (d, J=7.8 Hz, 1H), 7.64 (d, J=8.0 Hz, 1H), 7.49 (s, 2H), 7.37 (ddd, J=8.3, 7.3, 1.3 Hz, 1H), 7.26-7.10 (m, 3H). ¹³C NMR (101 MHz, DMSO-d₆): δ 171.68, 158.80, 156.41, 152.97, 150.62, 133.91, 130.35, 125.23 (2C), 122.36, 120.68 (2C), 120.53, 119.07, 115.00 (d, J=22.2 Hz). ¹⁹F NMR (376 MHz, DMSO-d₆): δ −119.72 (d, J=19.6 Hz). HPLC (λ₂₅₄): Purity: 96.0%; t_{R}: 8.980 min (HPLC analysis on a JASCO PU-2089/AS4050 apparatus with a Supelco analytical column Ascentis Express C18, 10 cm×4.6 mm, 5 μm, employing the following method: 5% B for 2 min, 5% B to 95% B over 9 min, 95% B for 5 min then from 95% B to 5% B over 1 min, 5% B for 3 min (total time: 20 min). Flow: 1 mL/min. Solvent A: water with 0.1% formic acid. Solvent B: acetonitrile with 0.1% formic acid).

MTF696: N-benzo[d]thiazol-2-yl-N″-(4-iodoaniline-carbonyl)-guanidine. In a 50 mL round-bottom flask, 1-(benzo[d]thiazol-2-yl) guanidine (500 mg, 2.60 mmol) was added to dioxane (25 mL). Once the compound had completely dissolved, 1-iodo-4-isocyanatobenzene (420 mg, 247 μL, 2.60 mmol) was introduced in one portion, leading to the progressive formation of a white precipitate. After completion of the reaction (TLC monitoring), the precipitate was filtered, then washed twice with technical grade ethanol (about 20 mL) to afford the titled compound as a white powder (347.8 mg, 41%). ¹H NMR (500 MHz, DMSO-d₆): δ 9.75 (s, 1H), 9.60 (s, 1H), 9.41 (s, 1H), 8.63 (s, 1H), 7.82 (d, J=7.9 Hz, 1H), 7.66 (dd, J=16.6, 8.2 Hz, 3H), 7.37 (t, J=7.7 Hz, 1H), 7.32 (s, 2H), 7.22 (t, J=7.5 Hz, 1H). ¹³C NMR (101 MHz, DMSO-d₆): δ 171.25, 152.96, 152.10, 150.53, 137.63, 137.01 (2C), 130.41, 125.23, 122.37, 121.36, 120.74 (2C), 119.12, 85.98. HPLC (λ254): Purity 99.1%; t_{R}: 10.330 min (HPLC analysis on a JASCO PU-2089/AS4050 apparatus with a Supelco analytical column Ascentis Express C18, 10 cm×4.6 mm, 5 μm, employing the following method: 5% B for 2 min, 5% B to 95% B over 9 min, 95% B for 5 min then from 95% B to 5% B over 1 min, 5% B for 3 min (total time: 20 min). Flow: 1 mL/min. Solvent A: water with 0.1% formic acid. Solvent B: acetonitrile with 0.1% formic acid).

MTF698: N-benzo[d]thiazol-2-yl-N″-(2,4-dimethoxyaniline-carbonyl)-guanidine. In a solution containing 2-amino-4H-benzo[4,5]thiazolo[3,2-a][1,3,5]triazin-4-one (200 mg, 916 μmol) and DBU (411 μL, 2.75 mmol) in dioxane (25 ml) is added 2,4-dimethoxyaniline (281 mg, 1.83 mmol). The mixture is heated under an inert atmosphere at 85° C. for 11 hours, then cooled down to r.t. and evaporated to dryness. Residual starting material was cleared by petroleum ether washings, then the residue was purified by silica gel flash chromatography using petroleum ether/AcOEt (8/2, v/v) to obtain a white solid (68.1 mg, 20%). ¹H NMR (500 MHz, DMSO-d₆): δ 10.14 (s, 1H), 9.65 (s, 1H), 9.38 (s, 1H), 8.20 (s, 1H), 7.97 (d, J=8.8 Hz, 1H), 7.84 (d, J=7.9 Hz, 1H), 7.64 (d, J=8.2 Hz, 1H), 7.37 (t, J=7.8 Hz, 1H), 7.23 (t, J=7.6 Hz, 1H), 6.68 (d, J=2.6 Hz, 1H), 6.52 (dd, J=8.9, 2.7 Hz, 1H), 3.95 (s, 3H), 3.76 (s, 3H). ¹³C NMR (101 MHz, DMSO-d₆): δ 171.46, 155.63, 152.98, 151.42, 151.03, 149.46, 130.65, 125.65, 122.87, 121.15, 120.34, 119.96, 119.55, 104.07, 98.67, 55.79, 55.15. HPLC (λ₂₅₄): Purity: 96.0%, t_{R}: 9.167 min (HPLC analysis on a JASCO PU-2089/AS4050 apparatus with a Supelco analytical column Ascentis Express C18, 10 cm×4.6 mm, 5 μm, employing the following method: 5% B for 2 min, 5% B to 95% B over 9 min, 95% B for 5 min then from 95% B to 5% B over 1 min, 5% B for 3 min (total time: 20 min). Flow: 1 mL/min. Solvent A: water with 0.1% formic acid. Solvent B: acetonitrile with 0.1% formic acid).

MTF719: N-benzo[d]thiazol-2-yl-N″-(4-methoxyethoxyaniline-carbonyl)-guanidine. In a solution containing 2-amino-4H-benzo[4,5]thiazolo[3,2-a][1,3,5]triazin-4-one (200 mg, 916 μmol) in dioxane (25 ml), DBU (411 μL, 2.75 mmol) and 4-(2-methoxyethoxy) aniline (351 μL, 1.83 mmol) were added. The mixture is heated under an inert atmosphere at 85° C. for 11 hours, then cooled down to r.t. and evaporated to dryness. Residual starting material was cleared by petroleum ether washings, then the residue was purified by silica gel flash chromatography using petroleum ether/AcOEt (8/2, v/v) to obtain a white solid (95 mg, 27%). ¹H NMR (400 MHz, DMSO-d₆): δ 9.69 (s, 1H), 9.34 (s, 2H), 9.01-8.54 (m, 1H), 7.79 (d, J=7.8 Hz, 1H), 7.62 (d, J=5.2 Hz, 1H), 7.35 (t, J=7.9 Hz, 3H), 7.24-7.15 (m, 1H), 6.92 (d, J=8.9 Hz, 2H), 4.07 (dd, J=10.2, 5.7 Hz, 2H), 3.68-3.61 (m, 2H), 3.31 (s, 3H). HPLC (λ₂₅₄): Purity: 98.2%, t_{R}: 8.397 min (HPLC analysis on a JASCO PU-2089/AS4050 apparatus with a Supelco analytical column Ascentis Express C18, 10 cm×4.6 mm, 5 μm, employing the following method: 5% B for 2 min, 5% B to 95% B over 9 min, 95% B for 5 min then from 95% B to 5% B over 1 min, 5% B for 3 min (total time: 20 min). Flow: 1 mL/min. Solvent A: water with 0.1% formic acid. Solvent B: acetonitrile with 0.1% formic acid).

### Comparative Compounds:

### N2,N2′-(disulfanediylbis(2,1-phenylene))bis(6-(trichloromethyl)-1,3,5-triazine-2,4-diamine) (CRO15)

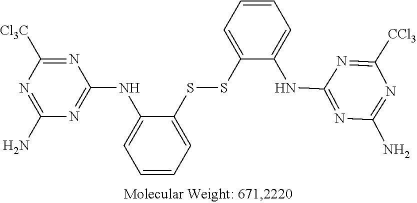

A solution of 1-(benzo[d]thiazol-2-yl) guanidine (10.0 g, 52 mmol) and trichloroacetonitrile (10.0 mL, 1.92 mmol) in technical grade ethanol (100 mL) was stirred at 75° C. After 1 h, a large amount of white precipitate appeared in the yellow solution. After reaction completion (TLC monitoring, about 3 h), the suspension was cold down to r.t. and filtered. The precipitate was washed with little amount of cold ethanol and dried at air. Recrystallization from acetone/diethyl ether afforded the desired compound as a white solid (10.03 g, 57.5%). TLC: R_{f} (Et₂O/PE, 1/1, v/v)=0.23. ¹H NMR (200 MHz, Acetone-d6): δ 8.54 (s, 1H), 7.96 (d, J=8.0 Hz, 1H), 7.58 (dd, J=7.8, 1.6 Hz, 1H), 7.35 (td, J=7.8, 1.6 Hz, 1H), 7.22-6.93 (m, 3H). ¹³C NMR (101 MHz, Acetone-d6): δ 174.00, 168.98, 166.37, 139.05, 133.76, 130.58, 129.92, 126.33, 125.34, 97.43. HRMS-ESI (m/z): [M+H]⁺ calc, for C₂₀H₁₅Cl₆N₁₀S₂⁺, 668.90482; Found: 668.90497. HPLC (>280): Purity 97.4%.

### N2,N2′-(disulfanediylbis(2,1-phenylene))bis(6-(3-ethoxyphenyl)-1,3,5-triazine-2,4-diamine) (MTF-319)

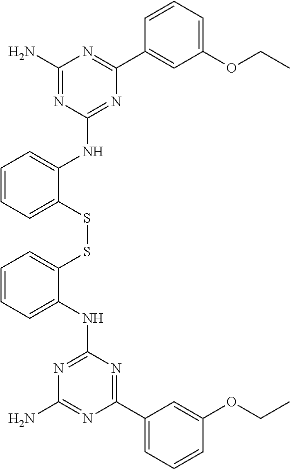 [SMILES: CCOc1cccc(-c2nc(N)nc(Nc3ccccc3SSc3ccccc3Nc3nc(N)nc(-c4cccc(OCC)c4)n3)n2)c1]

First, N—(N-(benzo[d]thiazol-2-yl) carbamimidoyl)-3-ethoxybenzimidamide was synthesized as follows: To a solution of 1-(benzo[d]thiazol-2-yl) guanidine (1.00 g, 5.20 mmol) in N,N-dimethylformamide (25 mL/g of guanidine) was added sodium hydride (60% dispersion in mineral oil, 1.5 eq., 312 mg, 7.81 mmol) and the mixture was stirred for 30 min at r.t. To this solution was added 3-ethoxybenzonitrile (765 mg, 5.20 mmol). The reaction was stirred overnight at room temperature and monitored by TLC. After completion of the reaction, the mixture was poured into water (200 mL/g of guanidine) and the precipitate was collected and washed with water, methanol and diethyl ether to afford the titled compound as a white powder (830 mg, 47%).

Then, a solution of N—(N-(benzo[d]thiazol-2-yl) carbamimidoyl)-3-ethoxybenzimidamide (339 mg, 1 mmol) in technical grade ethanol (approx. 25 mL/100 mg of biguanide) was stirred at reflux temperature and monitored by LCMS. After full conversion (approx 6-7 hours), the precipitate formed was filtered and washed with technical grade ethanol to afford the titled compound as a white powder (291 mg, 86%). ¹H NMR (200 MHz, DMSO-d6): δ 9.24 (s, 2H), 7.83 (d, J=8.8 Hz, 4H), 7.62 (dd, J=7.6, 1.1 Hz, 2H), 7.37 (t, J=7.8 Hz, 4H), 7.32-7.23 (m, 2H), 7.22-7.15 (m, 2H), 7.14-6.98 (m, 6H), 4.04 (q, J=6.9 Hz, 4H), 1.33 (t, J=6.9 Hz, 6H). ¹³C NMR (50 MHz, DMSO-d6): δ 169.98, 167.28, 165.48, 158.46, 138.12, 136.73, 133.68, 129.30, 128.39, 127.64, 126.61, 126.29, 120.13, 117.81, 113.27, 63.06, 14.68. HRMS-ESI (m/z): [M+H]⁺ calc. for C₃₄H₃₃N₁₀O₂S₂⁺, 677.22239; Found: 677.22253. HPLC (>280): Purity 100.0%.

### Reagents

Trypan blue, DMEM, penicillin/streptomycin and trypsin were purchased from Life Technology. Fetal calf serum (FCS) was purchased from HyClone. MCDB 153 medium was purchased from Sigma-Aldrich (Saint Quentin Fallavier, France).

### Cells

The BRAFV600E mutant melanoma line A375 (CRL-1619) was purchased from ATCC (Manassas, VA, USA). The A375 were maintained in glutamine-containing DMEM supplemented with 10% heat-inactivated fetal bovine serum, penicillin (100 units/ml) and streptomycin (50 mg/ml).

Normal human melanocytes were obtained from the foreskins of Caucasian children and grown in MCDB 153 medium supplemented with 2% fetal calf serum, 0.5 mg/ml hydrocortisone, 5 mg/ml insulin, 16 nM phorbol-12 myristate 13-acetate, 1 ng/ml basic fibroblast growth factor, 20 mg/ml bovine pituitary extract, 10 mM forskolin, and penicillin/streptomycin (100 U/ml/50 mg/ml). All cells were maintained in humidified incubators at 37° C. with 5% CO2.

### Cell Viability and Confluence Measurement

Cell viability was measured by the Trypan Blue exclusion method. Briefly, cells washed with PBS were harvested by a 3-minute incubation in 0.05% trypsin before neutralization with serum containing medium. An aliquot of the cell suspension was then mixed 1:1 with a 0.4% Trypan blue solution and viable cells were counted within 5 minutes on Kova slides under an optical microscope. Results are expressed as the mean percentage of control cells±SEM. N=3

Percentage of cell confluence was measured by IncuCyte® live-cell imaging (Essen Bioscience®, Ann Arbor, MI, USA]). Three wells were used per replicate and nine pictures per well were taken every hour for the indicated period of time using a 10× lens for each experiment. IncuCyte® software was used to analyze images. Results are represented as the mean confluence on all pictures±standard deviation.

### NCI 60 Assay

The effect of 10 μM of MTF504 was monitored on a panel 60 cell lines representing nine distinct tumour types: leukemia, colon, lung, central nervous system, renal, melanoma, ovarian, breast and prostate. Briefly, the compound was sent to the US National Cancer Institute where its antineoplastic effects were monitored on the panel of cell lines after 48 hours of treatment following a standardized protocol.

### DTP-NCI Assay

All compounds submitted to the NCI 60 Cell screen are tested initially at a single high dose (10-5 M) in the full NCI 60 cell panel. The results are reported as a mean of the percent growth of treated cells. The number reported is growth relative to the no-drug control, and relative to the time zero number of cells. This allows detection of both growth inhibition (values between 0 and 100).

| | | Viab | Viab | Viab | Viab | |
|---|---|---|---|---|---|---|
| | | A375 | A375 | A375 | A375 | |
| | | 10 μM/ | 5 μM/ | 10 μM/ | 5 μM/ | DTP- |
| ID | Structure | 24 h | 24 h | 48 h | 48 h | NCI |
| MTF214 | 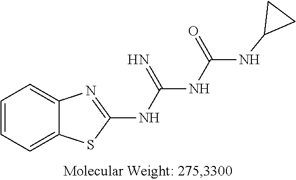 [SMILES: N=C(NC(=O)NC1CC1)Nc1nc2ccccc2s1] | 51.5% ±6.4 | 98.2% ±8.7 | 87.7% ±12.1 | 76.6% ±5.1 | |
| MTF314 | 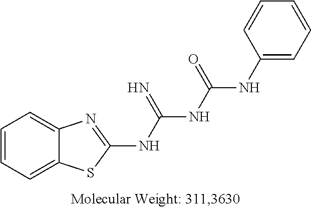 [SMILES: N=C(NC(=O)Nc1ccccc1)Nc1nc2ccccc2s1] | 32.7% ±4.7 | 53.5% ±19.6 | 7.6% ±4.5 | 12.3% ±12.3 | 37.41 |
| MTF415 | 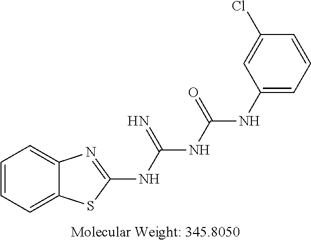 [SMILES: N=C(NC(=O)Nc1cccc(Cl)c1)Nc1nc2ccccc2s1] | 58.3% ±8.7 | 93.5% ±6.7 | 19.9% ±11.9 | 32.9% ±12.1 | 35.38 |
| MTF418 | 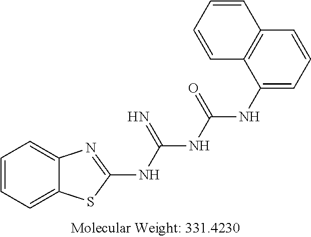 [SMILES: N=C(NC(=O)Nc1cccc2ccccc12)Nc1nc2ccccc2s1] | 41.0% ±1.4 | 16.5% ±1.5 | 4.6% ±10.7 | 16.7% ±2.8 | 26.06 |
| MTF419 | 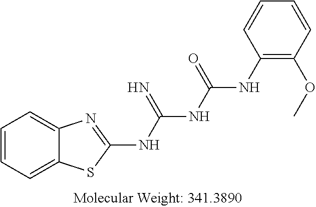 [SMILES: COc1ccccc1NC(=O)NC(=N)Nc1nc2ccccc2s1] | 56.8% ±7.0 | 62.6% ±4.2 | 21.8% ±8.1 | 18.8% ±1.0 | 26.69 |
| MTF420 | 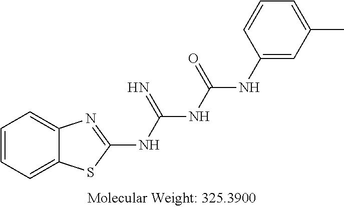 [SMILES: Cc1cccc(NC(=O)NC(=N)Nc2nc3ccccc3s2)c1] | 48.9% ±3.5 | 59.0% ±11.6 | 20.4% ±8.7 | 24.8% ±1.2 | 32.89 |
| MTF426 | 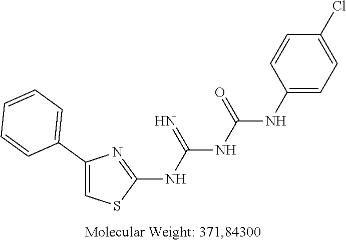 [SMILES: N=C(NC(=O)Nc1ccc(Cl)cc1)Nc1nc(-c2ccccc2)cs1] | | 109.2% ±1.4 | | 46.4% ±6.6 | |
| MTF427 | 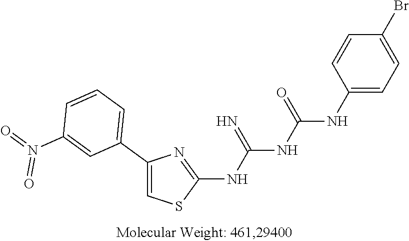 [SMILES: N=C(NC(=O)Nc1ccc(Br)cc1)Nc1nc(-c2cccc([N+](=O)[O-])c2)cs1] | | 94.2% ±18.4 | | 34.4% ±4.6 | 72.55 |
| MTF428 | 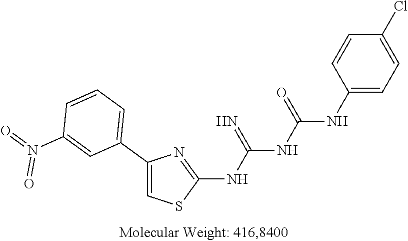 [SMILES: N=C(NC(=O)Nc1ccc(Cl)cc1)Nc1nc(-c2cccc([N+](=O)[O-])c2)cs1] | | 127.5% ±11.3 | | 50.2% ±2.0 | |
| MTF429 | 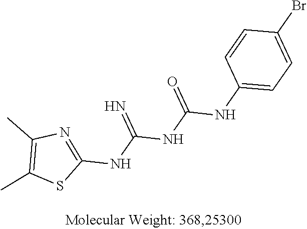 [SMILES: Cc1nc(NC(=N)NC(=O)Nc2ccc(Br)cc2)sc1C] | | 94.2% ±7.1 | | 39.1% ±13.9 | |
| MTF430 | 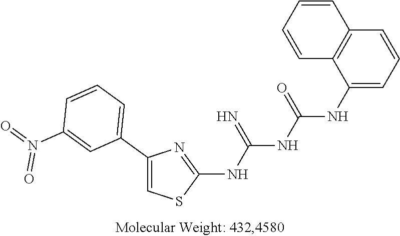 [SMILES: N=C(NC(=O)Nc1cccc2ccccc12)Nc1nc(-c2cccc([N+](=O)[O-])c2)cs1] | | 75.8% ±15.6 | | 25.3% ±7.2 | 89.23 |
| MTF431 | 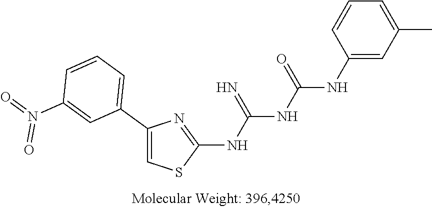 [SMILES: Cc1cccc(NC(=O)NC(=N)Nc2nc(-c3cccc([N+](=O)[O-])c3)cs2)c1] | | 95.4% ±20.5 | | 41.6% ±4.6 | |
| MTF432 | 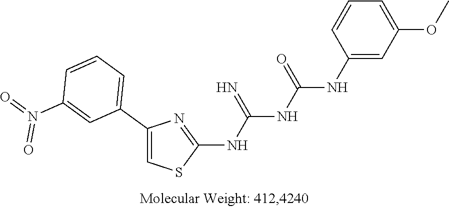 [SMILES: COc1cccc(NC(=O)NC(=N)Nc2nc(-c3cccc([N+](=O)[O-])c3)cs2)c1] | | 115% ±1.4 | | 29.8% ±3.5 | 74.83 |
| MTF433 | 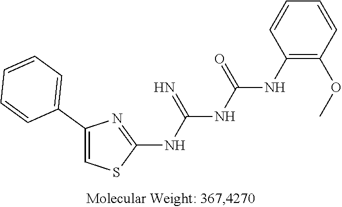 [SMILES: COc1ccccc1NC(=O)NC(=N)Nc1nc(-c2ccccc2)cs1] | | 109.4% ±2.5 | | 43.9% ±3.0 | |
| MTF434 | 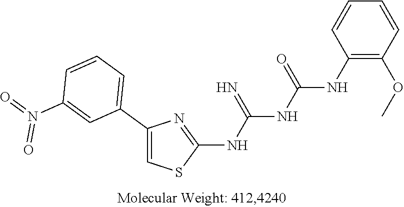 [SMILES: COc1ccccc1NC(=O)NC(=N)Nc1nc(-c2cccc([N+](=O)[O-])c2)cs1] | | 63.8% ±10.6 | | 50.7% ±3.8 | |
| MTF435 | 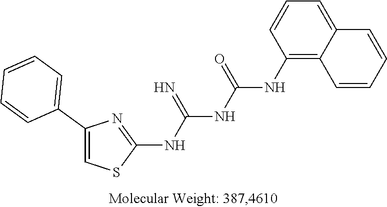 [SMILES: N=C(NC(=O)Nc1cccc2ccccc12)Nc1nc(-c2ccccc2)cs1] | | 81.0% ±2.8 | | 84.6% ±1.4 | |
| MTF495 | 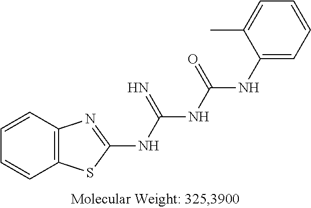 [SMILES: Cc1ccccc1NC(=O)NC(=N)Nc1nc2ccccc2s1] | | 38.6% ±5.8 | | 8.0% ±4.4 | 25.90 |
| MTF496 | 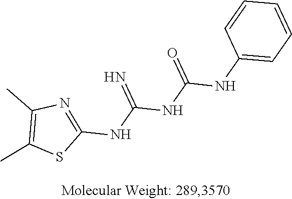 [SMILES: Cc1nc(NC(=N)NC(=O)Nc2ccccc2)sc1C] | | 67.7% ±10.5 | | 56.6% ±2.1 | 83.79 |
| MTF504 | 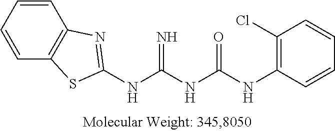 [SMILES: N=C(NC(=O)Nc1ccccc1Cl)Nc1nc2ccccc2s1] | | 26.0% ±5.9 | | 7.1% ±6.0 | 25.42 5 doses |
| MTF505 | 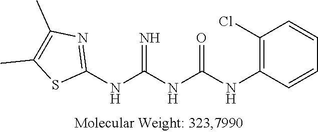 [SMILES: Cc1nc(NC(=N)NC(=O)Nc2ccccc2Cl)sc1C] | | 31.1% ±8.5 | | 10.0% ±1.5 | 33.00 5 doses |
| MTF506 | 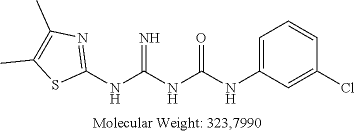 [SMILES: Cc1nc(NC(=N)NC(=O)Nc2cccc(Cl)c2)sc1C] | | 64.3% ±19.1 | | 54.5% ±13.4 | 67.74 |
| MTF507 | 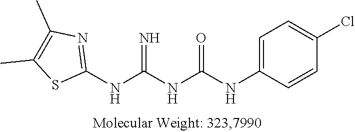 [SMILES: Cc1nc(NC(=N)NC(=O)Nc2ccc(Cl)cc2)sc1C] | | 98.9% ±12.0 | | 138.8% ±32.5 | |
| MTF509 | 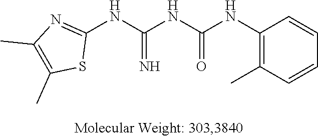 [SMILES: Cc1ccccc1NC(=O)NC(=N)Nc1nc(C)c(C)s1] | | 23.0% ±13.9 | | 13.6% ±1.0 | 29.33 5 doses |
| MTF510 | 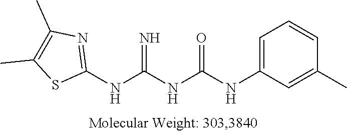 | | 56.5% ±9.6 | | 26.1% ±3.5 | 47.18 |
| MTF511 | 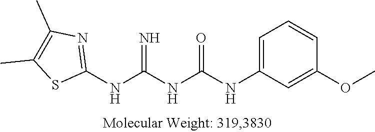 | | 92.1% ±1.4 | | 82.8% ±9.2 | |
| MTF512 | 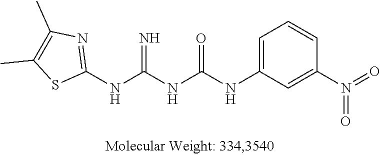 [SMILES: Cc1nc(NC(=N)NC(=O)Nc2cccc([N+](=O)[O-])c2)sc1C] | | 87.0% ±7.1 | | 54.3% ±7.1 | |
| MTF515 | 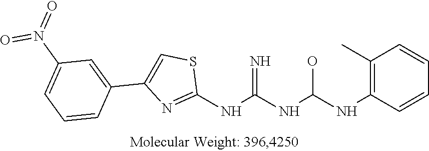 [SMILES: Cc1ccccc1NC(O)NC(=N)Nc1nc(-c2cccc([N+](=O)[O-])c2)cs1] | | 64.9% ±16.2 | | 28.7% ±16.8 | 37.72 |
| MTF649 | 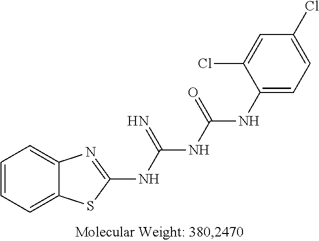 [SMILES: N=C(NC(=O)Nc1ccc(Cl)cc1Cl)Nc1nc2ccccc2s1] | | | | | −58.23 5 doses (×2) |
| MTF650 | 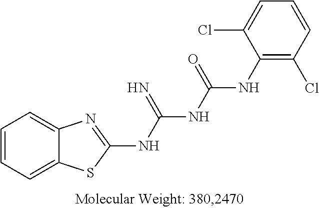 [SMILES: N=C(NC(=O)Nc1c(Cl)cccc1Cl)Nc1nc2ccccc2s1] | | | | | 36.41 |
| MTF651 | 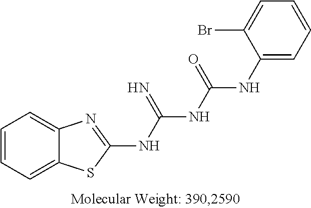 [SMILES: N=C(NC(=O)Nc1ccccc1Br)Nc1nc2ccccc2s1] | | | | | −17.80 5 doses (×2) |
| MTF652 | 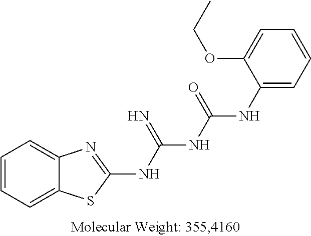 [SMILES: CCOc1ccccc1NC(=O)NC(=N)Nc1nc2ccccc2s1] | | | | | 12.58 5 doses |
| MTF653 | 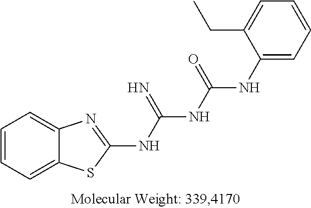 [SMILES: CCc1ccccc1NC(=O)NC(=N)Nc1nc2ccccc2s1] | | | | | 0.27 5 doses |
| MTF654 | 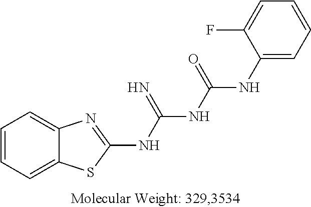 [SMILES: N=C(NC(=O)Nc1ccccc1F)Nc1nc2ccccc2s1] | | | | | 20.33 5 doses |
| MTF655 | 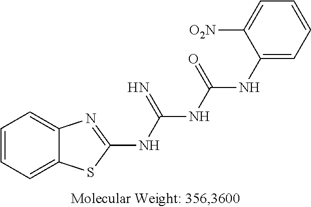 [SMILES: N=C(NC(=O)Nc1ccccc1[N+](=O)[O-])Nc1nc2ccccc2s1] | | | | | 11.59 5 doses |
| MTF656 | 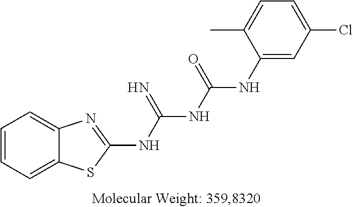 [SMILES: Cc1ccc(Cl)cc1NC(=O)NC(=N)Nc1nc2ccccc2s1] | | | | | −19.42 5 doses (×2) |
| MTF657 | 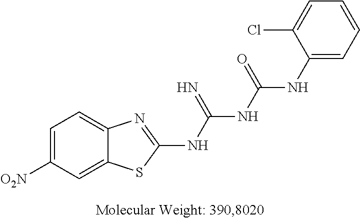 [SMILES: N=C(NC(=O)Nc1ccccc1Cl)Nc1nc2ccc([N+](=O)[O-])cc2s1] | | | | | 31.16 5 doses |

1-12. (canceled)

13. A compound of formula (I) 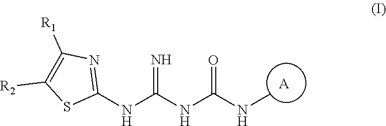
wherein
R₁ and R₂ are independently selected from H, C₁-C₆ alkyl, C₃-C₆ cycloalkyl, C₁-C₆ haloalkyl, C₂-C₆ alkenyl, C₂-C₆ alkynyl, heterocyclyl having 5 to 10 ring atoms, aryl having 6 to 10 ring atoms, heteroaryl having 5 to 10 ring atoms, and C₇-C₁₆ aralkyl, said alkyl, cycloalkyl, haloalkyl, alkenyl, alkynyl, heterocyclyl, aryl, heteroaryl and aralkyl being optionally substituted with one or more substituents preferably selected from oxo, halogen, C₁-C₆ alkyl, C₁-C₆ alkoxy, C₁-C₆ haloalkyl, —OH, —OR, —NR′R″, —NO₂, —CN and —(CO)—R;
or R₁ and R₂, together with the carbon-carbon double bond between them, form a 6 to 10 membered aryl or heteroaryl ring, said aryl and heteroaryl being optionally substituted with one or more substituents preferably selected from oxo, halogen, C₁-C₆ alkyl, C₁-C₆ alkoxy, C₁-C₆ haloalkyl, —OH, —OR, —NR′R″, —NO₂, —CN and —(CO)—R;
Ring A is selected from aryl having 6 to 10 ring atoms, heteroaryl having 5 to 10 ring atoms, C₃-C₆ cycloalkyl, and heterocyclyl having 5 to 10 ring atoms, said aryl, heteroaryl, cycloalkyl and heterocyclyl being optionally substituted with one or more substituents preferably selected from oxo, halogen, C₁-C₆ alkyl, C₁-C₆ alkenyl, C₁-C₆ alkynyl, C₁-C₆ alkoxy, C₁-C₆ haloalkyl, —OH, —OR, —NR′R″, —NO₂, —CN and —(CO)—R;
each R is independently selected from H, C₁-C₆ alkyl, OH, C₁-C₆ alkoxy and —NR′R″; and
each R′ and R″ is independently selected from H and C₁-C₆ alkyl, and pharmaceutically acceptable salts thereof.

14. The compound according to claim 13, wherein the compound is of formula (II), (III) or (IV) 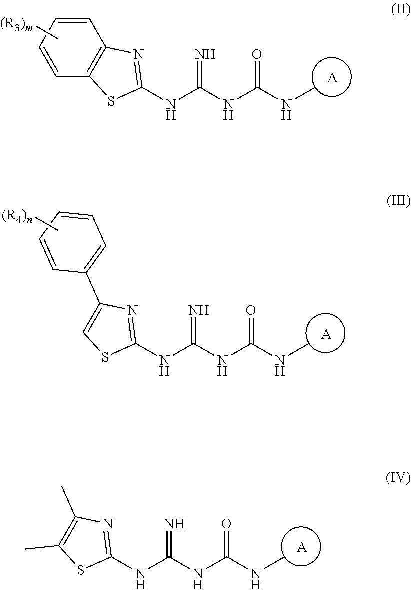
wherein
m is an integer between 0 and 5, preferably m is 0 or 1;
R₃ is independently selected from halogen, C₁-C₆ alkyl, C₁-C₆ alkoxy, C₁-C₆ haloalkyl, —OH, —NR′R″, —NO₂, —CN and —(CO)—R;
n is an integer between 0 and 5, preferably n is 0 or 1;
R₄ is independently selected from halogen, C₁-C₆ alkyl, C₁-C₆ alkoxy, C₁-C₆ haloalkyl, —OH, —NR′R″, —NO₂, —CN and —(CO)—R; and
Ring A, R, R′ and R″ are as defined in claim 13.

15. The compound of formula (II) according to claim 14, wherein m is 0 or 1, and when m is 1, R₃ is —NO₂.

16. The compound of formula (III) according to claim 14, wherein n is 0 or 1, and when n is 1, R₄ is —NO₂.

17. The compound according to claim 13, wherein Ring A is a phenyl or a naphthyl, said phenyl and naphthyl being optionally substituted with one or more substituents preferably selected from halogen, C₁-C₆ alkyl, C₁-C₆ alkoxy, and —NO₂.

18. The compound according to claim 13, wherein said compound is selected from

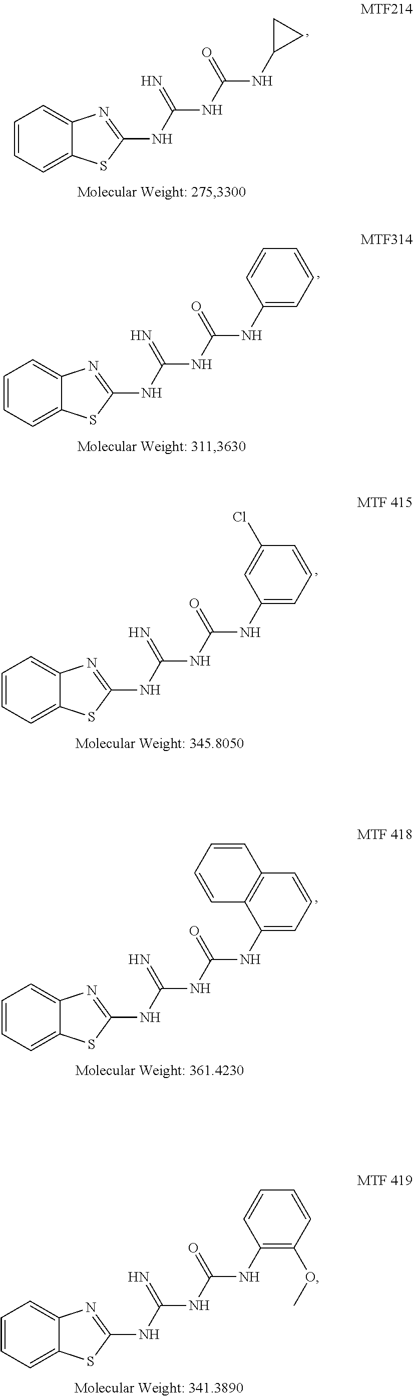 [SMILES: N=C(NC(=O)NC1CC1)Nc1nc2ccccc2s1 ; N=C(NC(=O)Nc1ccccc1)Nc1nc2ccccc2s1 ; N=C(NC(=O)Nc1cccc(Cl)c1)Nc1nc2ccccc2s1 ; N=C(NC(=O)Nc1cccc2ccccc12)Nc1nc2ccccc2s1 ; COc1ccccc1NC(=O)NC(=N)Nc1nc2ccccc2s1]

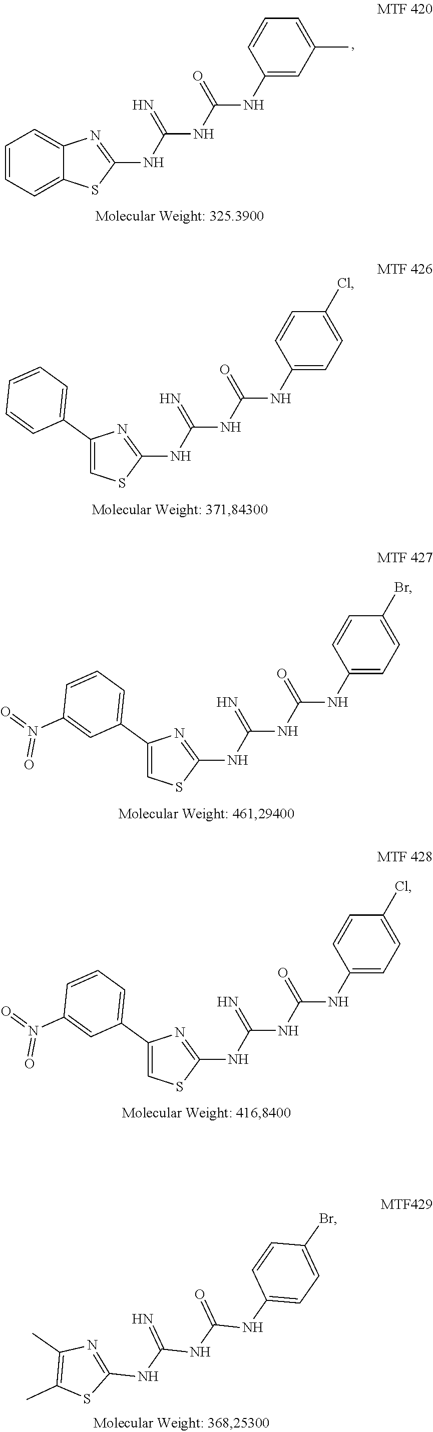

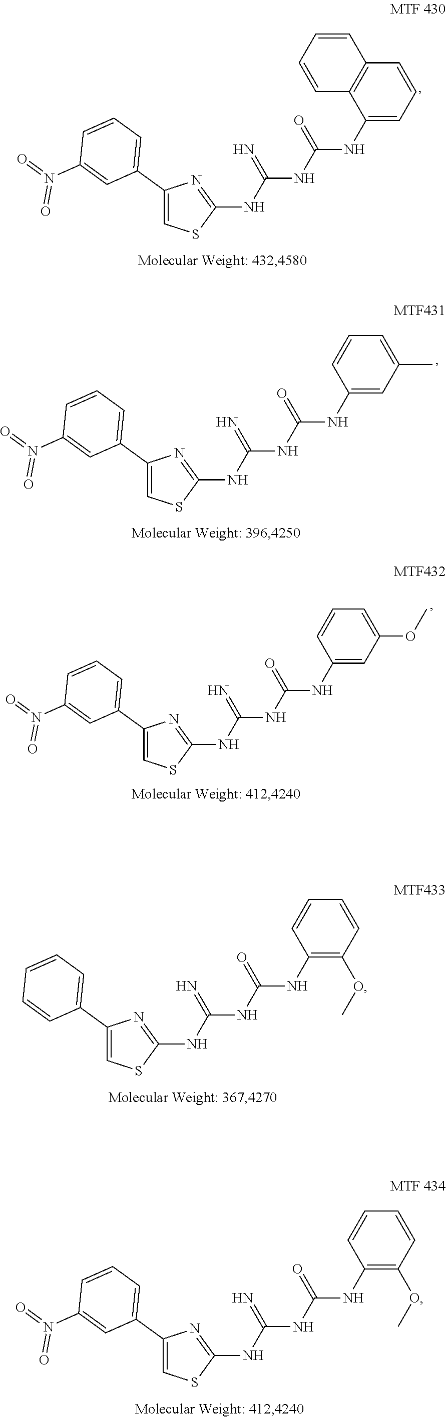 [SMILES: N=C(NC(=O)Nc1cccc2ccccc12)Nc1nc(-c2cccc([N+](=O)[O-])c2)cs1 ; Cc1cccc(NC(=O)NC(=N)Nc2nc(-c3cccc([N+](=O)[O-])c3)cs2)c1 ; COc1cccc(NC(=O)NC(=N)Nc2nc(-c3cccc([N+](=O)[O-])c3)cs2)c1 ; COc1ccccc1NC(=O)NC(=N)Nc1nc(-c2ccccc2)cs1 ; COc1ccccc1NC(=O)NC(=N)Nc1nc(-c2cccc([N+](=O)[O-])c2)cs1]

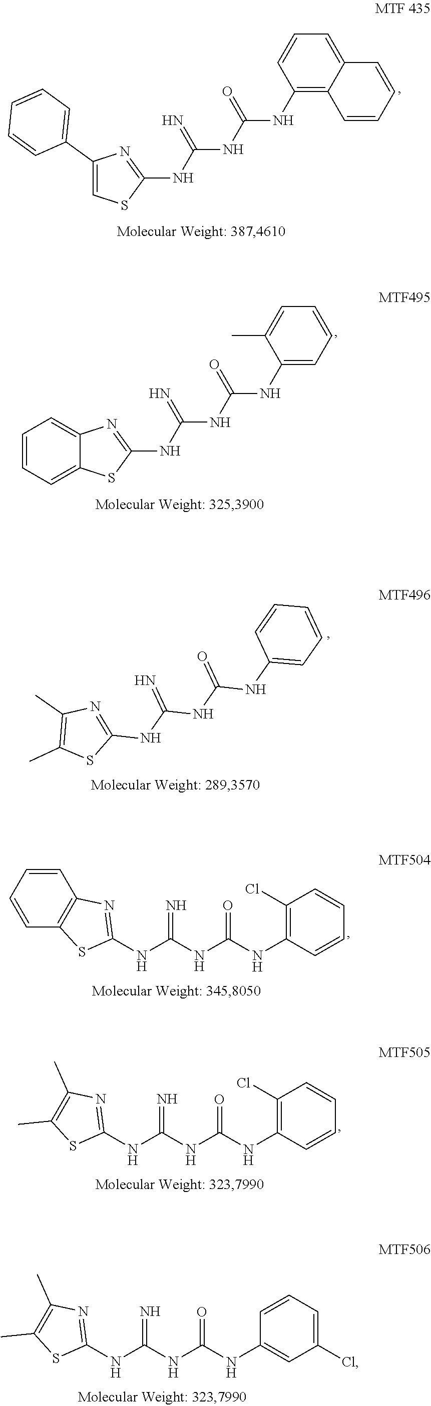 [SMILES: N=C(NC(=O)Nc1cccc2ccccc12)Nc1nc(-c2ccccc2)cs1 ; Cc1ccccc1NC(=O)NC(=N)Nc1nc2ccccc2s1 ; Cc1nc(NC(=N)NC(=O)Nc2ccccc2)sc1C ; N=C(NC(=O)Nc1ccccc1Cl)Nc1nc2ccccc2s1 ; Cc1nc(NC(=N)NC(=O)Nc2ccccc2Cl)sc1C ; Cc1nc(NC(=N)NC(=O)Nc2cccc(Cl)c2)sc1C]

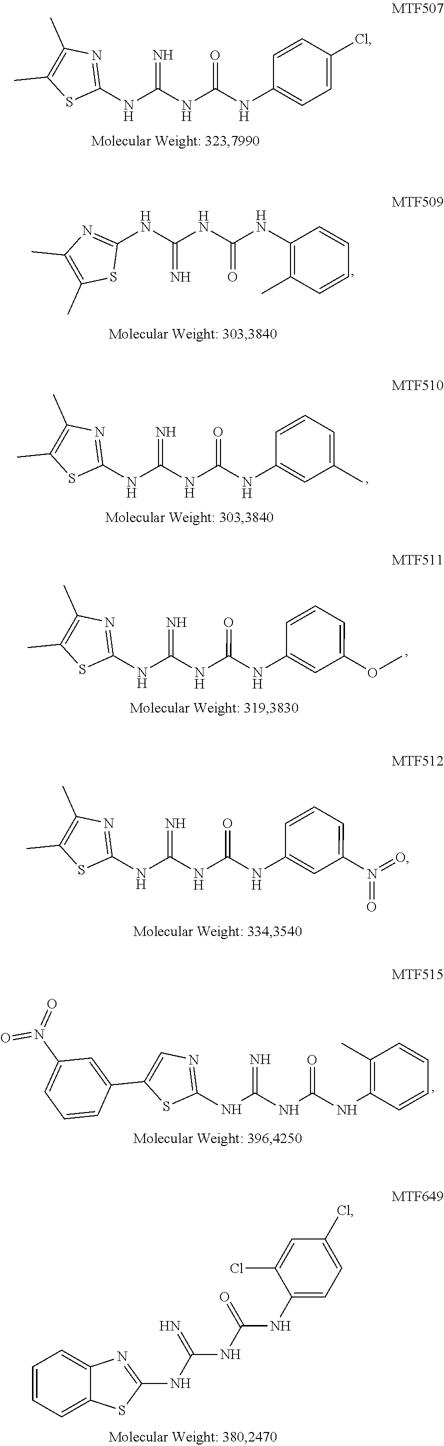

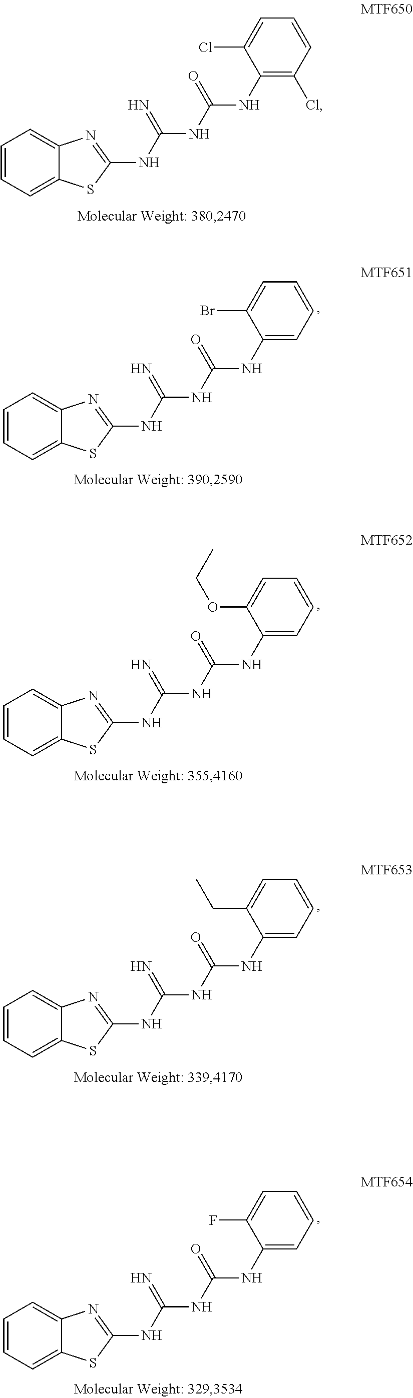 [SMILES: N=C(NC(=O)Nc1c(Cl)cccc1Cl)Nc1nc2ccccc2s1 ; N=C(NC(=O)Nc1ccccc1Br)Nc1nc2ccccc2s1 ; CCOc1ccccc1NC(=O)NC(=N)Nc1nc2ccccc2s1 ; CCc1ccccc1NC(=O)NC(=N)Nc1nc2ccccc2s1 ; N=C(NC(=O)Nc1ccccc1F)Nc1nc2ccccc2s1]

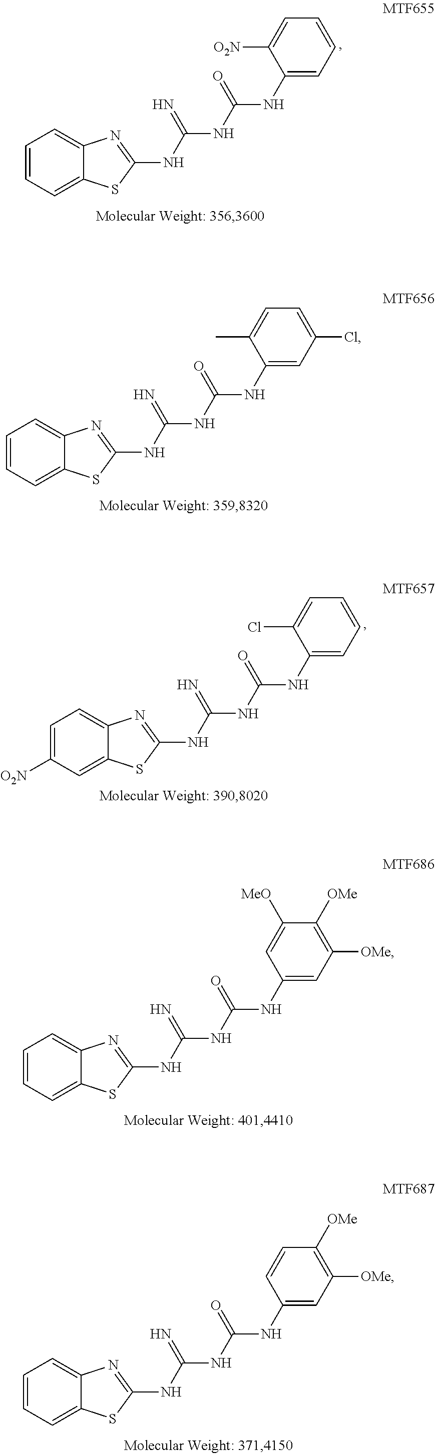 [SMILES: N=C(NC(=O)Nc1ccccc1[N+](=O)[O-])Nc1nc2ccccc2s1 ; Cc1ccc(Cl)cc1NC(=O)NC(=N)Nc1nc2ccccc2s1 ; N=C(NC(=O)Nc1ccccc1Cl)Nc1nc2ccc([N+](=O)[O-])cc2s1 ; COc1cc(NC(=O)NC(=N)Nc2nc3ccccc3s2)cc(OC)c1OC ; COc1ccc(NC(=O)NC(=N)Nc2nc3ccccc3s2)cc1OC]

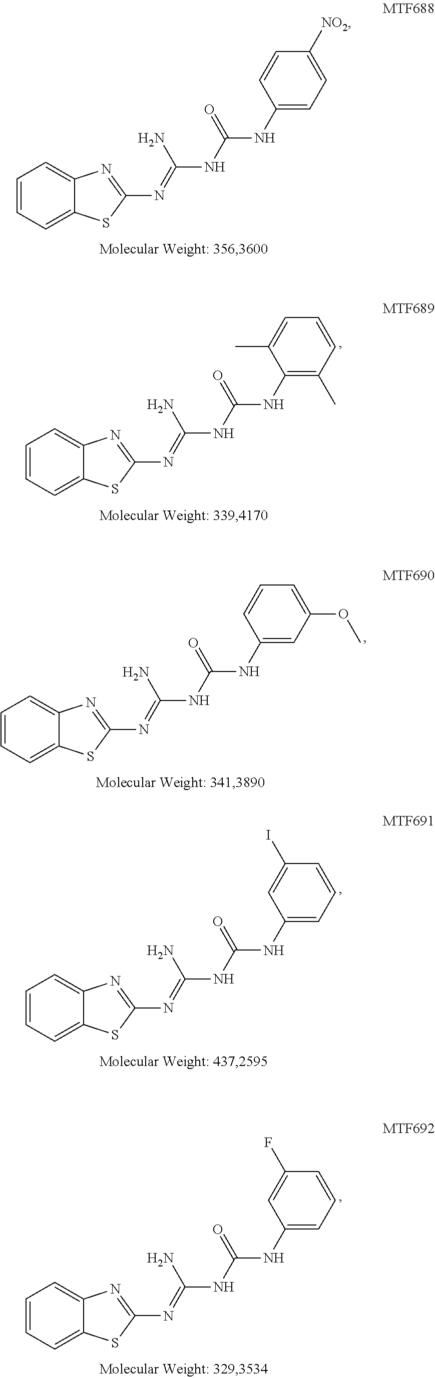 [SMILES: N/C(=N\c1nc2ccccc2s1)NC(=O)Nc1ccc([N+](=O)[O-])cc1 ; Cc1cccc(C)c1NC(=O)N/C(N)=N/c1nc2ccccc2s1 ; COc1cccc(NC(=O)N/C(N)=N/c2nc3ccccc3s2)c1 ; N/C(=N\c1nc2ccccc2s1)NC(=O)Nc1cccc(I)c1 ; N/C(=N\c1nc2ccccc2s1)NC(=O)Nc1cccc(F)c1]

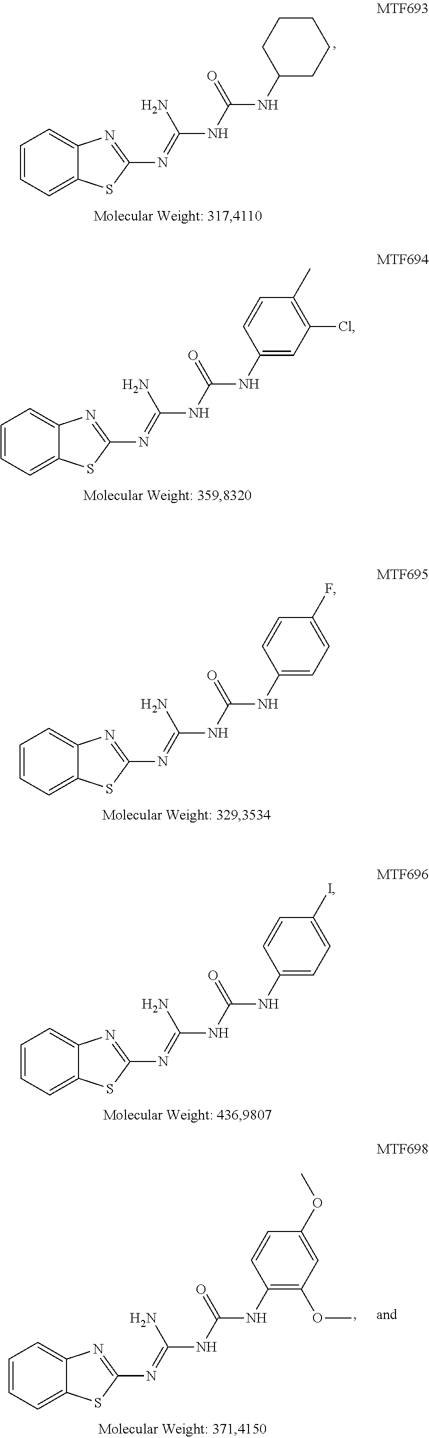 [SMILES: N/C(=N\c1nc2ccccc2s1)NC(=O)NC1CCCCC1 ; Cc1ccc(NC(=O)N/C(N)=N/c2nc3ccccc3s2)cc1Cl ; N/C(=N\c1nc2ccccc2s1)NC(=O)Nc1ccc(F)cc1 ; N/C(=N\c1nc2ccccc2s1)NC(=O)Nc1ccc(I)cc1 ; COc1ccc(NC(=O)N/C(N)=N/c2nc3ccccc3s2)c(OC)c1]

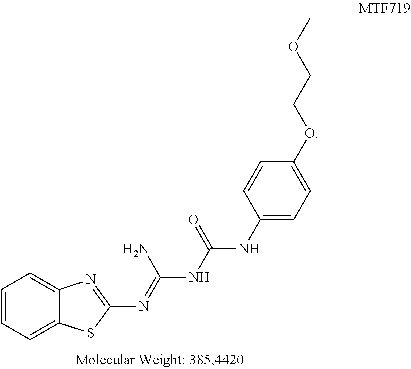 [SMILES: COCCOc1ccc(NC(=O)N/C(N)=N/c2nc3ccccc3s2)cc1]

19. The compound according to claim 13, wherein said compound is selected from

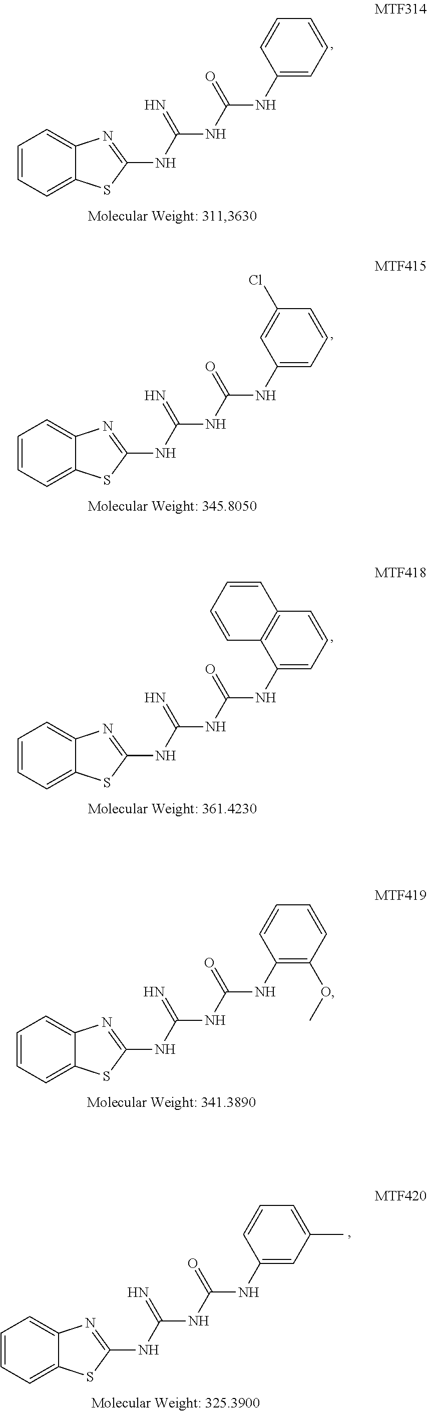 [SMILES: N=C(NC(=O)Nc1ccccc1)Nc1nc2ccccc2s1 ; N=C(NC(=O)Nc1cccc(Cl)c1)Nc1nc2ccccc2s1 ; N=C(NC(=O)Nc1cccc2ccccc12)Nc1nc2ccccc2s1 ; COc1ccccc1NC(=O)NC(=N)Nc1nc2ccccc2s1 ; Cc1cccc(NC(=O)NC(=N)Nc2nc3ccccc3s2)c1]

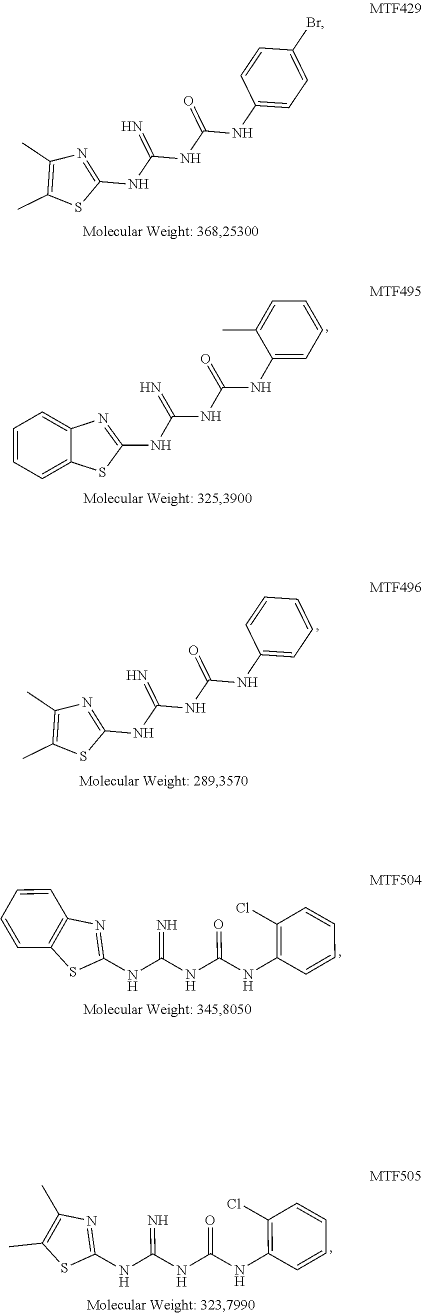 [SMILES: Cc1nc(NC(=N)NC(=O)Nc2ccc(Br)cc2)sc1C ; Cc1ccccc1NC(=O)NC(=N)Nc1nc2ccccc2s1 ; Cc1nc(NC(=N)NC(=O)Nc2ccccc2)sc1C ; N=C(NC(=O)Nc1ccccc1Cl)Nc1nc2ccccc2s1 ; Cc1nc(NC(=N)NC(=O)Nc2ccccc2Cl)sc1C]

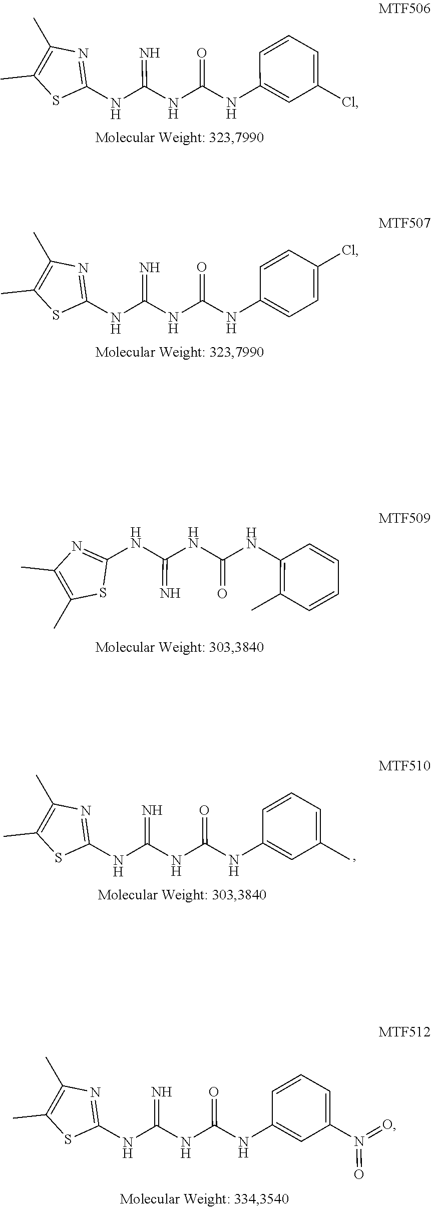

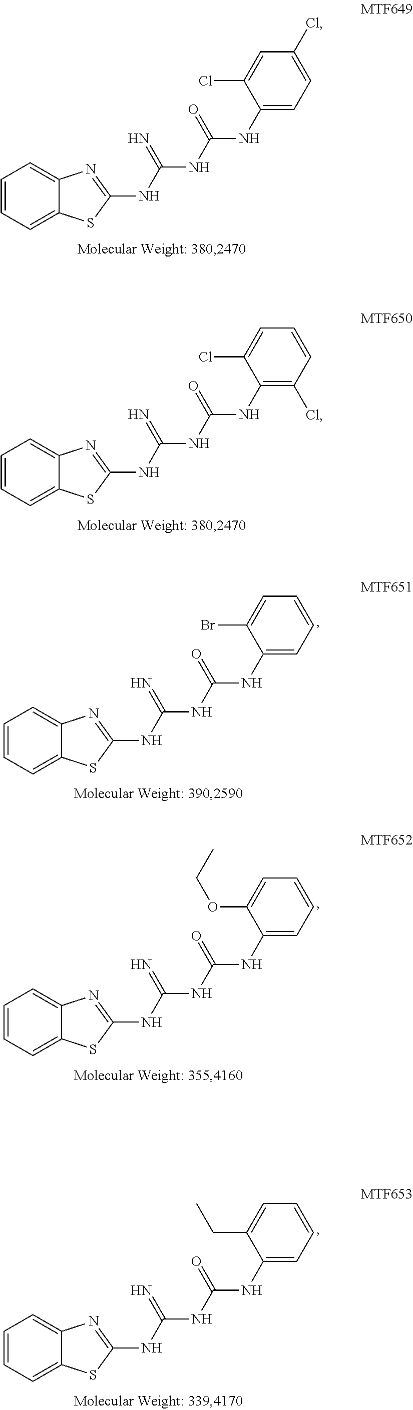 [SMILES: N=C(NC(=O)Nc1ccc(Cl)cc1Cl)Nc1nc2ccccc2s1 ; N=C(NC(=O)Nc1c(Cl)cccc1Cl)Nc1nc2ccccc2s1 ; N=C(NC(=O)Nc1ccccc1Br)Nc1nc2ccccc2s1 ; CCOc1ccccc1NC(=O)NC(=N)Nc1nc2ccccc2s1 ; CCc1ccccc1NC(=O)NC(=N)Nc1nc2ccccc2s1]

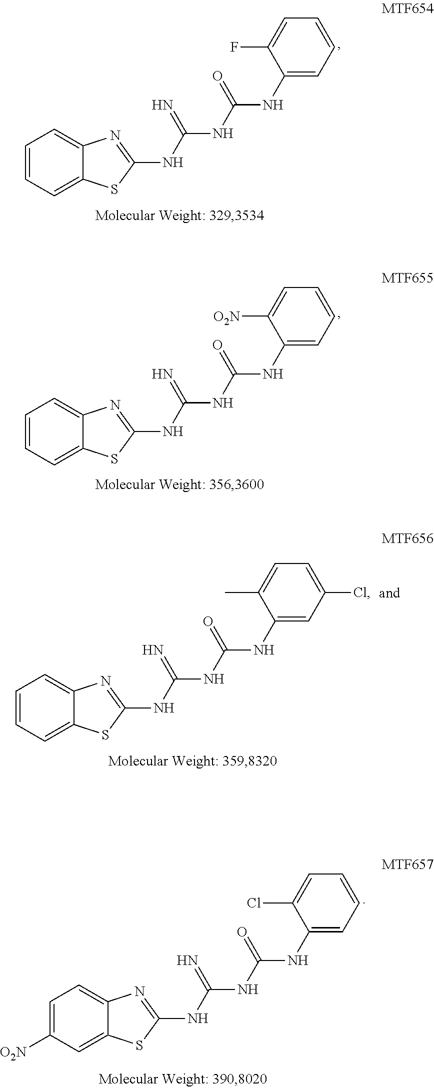 [SMILES: N=C(NC(=O)Nc1ccccc1F)Nc1nc2ccccc2s1 ; N=C(NC(=O)Nc1ccccc1[N+](=O)[O-])Nc1nc2ccccc2s1 ; Cc1ccc(Cl)cc1NC(=O)NC(=N)Nc1nc2ccccc2s1 ; N=C(NC(=O)Nc1ccccc1Cl)Nc1nc2ccc([N+](=O)[O-])cc2s1]

20. A pharmaceutical composition comprising a compound according to claim 13, and a pharmaceutically acceptable carrier.

21. A compound according to claim 13, for use as a drug.

22. The compound for use according to claim 21, for use in a method for treating cancer, preferably selected from leukemia, Non-Small Cell Lung Cancer (NSCLC), colon cancer, central nervous system (CNS) cancer, melanoma, ovarian cancer, renal cancer, prostate and breast cancer.

23. The compound for use according to claim 21, for use in a method for treating melanoma.

24. The compound for use according to claim 21, for use in a method for treating BRAF inhibitor-resistant melanoma.
